(19) United States
(12) Patent Application Publication
(10) Pub. No.: US 2017/0370836 A1
(43) Pub. Date: Dec. 28, 2017

(54) MOBILE/WEARABLE DEVICES INCORPORATING LSPR SENSORS

(71) Applicant: LamdaGen Corporation, Menlo Park, CA (US)

(72) Inventors: Daniele Gerion, Oakland, CA (US); Randolph Storer, Hillsborough, CA (US)

(21) Appl. No.: 15/618,670

(22) Filed: Jun. 9, 2017

Related U.S. Application Data
(63) Continuation of application No. PCT/US2015/000410, filed on Dec. 23, 2015.
(60) Provisional application No. 62/096,785, filed on Dec. 24, 2014.

(51) Int. Cl. G01N 21/552; G01N 21/25; G01N 33/543; G01N 21/82

(52) U.S. Cl. CPC G01N 21/554; G01N 33/543; G01N 21/253; G01N 21/82; G01N 2201/0221

(57) ABSTRACT

Sensor chips and devices that incorporate localized surface plasmon resonance (LSPR) sensors are described which are suitable for use in near-patient and point-of-care diagnostic testing. In some embodiments, LSPR sensors are integrated with microfabricated fluidics and other system components to create compact, portable bench-top or hand-held diagnostic testing systems. In some embodiments, all components are packaged in compact, portable wearable devices.

### CROSS-REFERENCE

This application claims the benefit of U.S. Provisional Application No. 62/096,785, filed Dec. 24, 2014, which application is incorporated herein in its entirety by reference.

### BACKGROUND OF THE INVENTION

At present, most clinical diagnostic testing is performed in central laboratories using high-throughput, automated assay platform technologies that make use of direct optical detection (e.g. absorbance), indirect (label-based) optical detection (e.g. radioisotopes, fluorophores, quantum dots), or electrochemical detection (e.g. using voltammetry or amperometry to detect redox reactions) to identify and quantify the presence of biological molecules (analytes) in complex biological samples with high sensitivity and high specificity.

Over the past 20 years, there has been an effort to move some types of clinical diagnostic testing out of the central laboratories to the near-patient or point-of-care settings. Testing of patients by primary care health providers at the point-of-care has been shown, for some types of test, to provide faster time-to-results, improved diagnosis and treatment decisions, better patient compliance with recommended treatment regimes, and improved healthcare outcomes.

Current point-of-care diagnostic instruments are generally limited to qualitatively detecting whether or not an analyte is present. They generally lack sensitivity and are often not able to quantify the amount of analyte present. One example of a point-of-care diagnostics instrument that can provide quantitative data is the i-STAT (currently owned by Abbott Point-of-Care), a hand-held device that utilizes disposable test cartridges that include on-board assay reagents and fluidics to process very small volumes of sample (e.g. blood) and provide sample-to-answer test results in minutes. A menu of test cartridges is available, where the type of test performed is determined by the choice of test cartridge. However, the i-STAT lacks the sensitivity required for many types of assays. Furthermore, many if not all i-STAT tests utilize electrochemical detection to identify and quantify the presence of biological markers (e.g. proteins, small molecules, ions, etc.) in the sample.

More recently, there has been an effort to harness the rapid advance of mobile phone technology for remote monitoring and healthcare applications. A number of cell-phone apps and wearable devices have reached the market, including pedometers, heart rate monitors, activity/sleep sensors, etc. To date, most of these devices are for non-clinical applications, but there is growing interest in pushing these types of technologies into the clinical diagnostics testing space, i.e. to develop devices that are wearable and/or handheld and that perform the types of biochemical assays used in clinical diagnostics testing.

One component of such devices will be sensors that can be mass produced at low cost and are sensitive, reproducible, and robust enough to meet the requirements for clinical testing.

### SUMMARY

In some embodiments of the present disclosure, a sensor chip may comprise one or more reaction wells, wherein each reaction well comprises a sensor surface capable of sustaining a localized surface plasmon resonance. The sensor chip may also comprise a sample reservoir configured to contain a sample comprising an analyte. The sensor chip may also comprise one or more fluid conduits, wherein each fluid conduit connects the sample reservoir and one of the reaction wells. The one or more sensor surfaces may exhibit an analyte-induced change in optical property upon contact with the sample.

A device may comprise one or more sensor chips, wherein a sensor chip comprises one or more sensor surfaces, wherein each sensor surface is capable of sustaining a localized surface plasmon resonance, and wherein each sensor surface is contained within a reaction well. The sensor chip may also comprise a sample reservoir configured to contain a sample comprising an analyte. The sensor chip may also comprise one or more fluid conduits, wherein each fluid conduit connects the sample reservoir and one of the reaction wells. The device may also comprise one or more light sources configured to illuminate the one or more sensor surfaces. The device may also comprise one or more detectors configured to detect an analyte-induced change in an optical property of light reflected or transmitted by the one or more sensor surfaces.

A device may comprise one or more light sources configured to illuminate one or more sensor surfaces on a sensor chip. The device may also comprise one or more detectors configured to detect an analyte-induced change in an optical property of light reflected or transmitted by the one or more sensor surfaces. The device may also contain a piston configured to couple with a reservoir on the sensor chip to actuate flow of sample from the reservoir onto the one or more sensor surfaces.

A device may comprise one or more sensor chips, wherein a sensor chip comprises one or more sensor surfaces, wherein each sensor surface is capable of sustaining a localized surface plasmon resonance, and wherein each sensor is contained within a reaction well. The sensor chip may also comprise an optical system configured to capture images and detect an analyte induced change in an optical property of light reflected or transmitted by the one or more sensor surfaces. The device may also comprise a processor for processing the images and determining a concentration of the analyte based on analysis of a series of two or more images. The analyte change may be detected in one or more corresponding pixels in the series of two or more images near locations where analyte molecules are bound to the one or more sensor surfaces.

Disclosed herein are sensor chips comprising: (a) one or more reaction wells, wherein each reaction well comprises a sensor surface capable of sustaining a localized surface plasmon resonance; (b) a sample reservoir configured to contain a sample comprising an analyte; and (c) one or more fluid conduits, wherein each fluid conduit connects the sample reservoir and one of the reaction wells; wherein the one or more sensor surfaces exhibit an analyte-induced change in optical property upon contact with the sample.

In some embodiments, the sensor chip may further comprise a primary binding component immobilized on each of the one or more sensor surface(s), wherein the primary binding component is selected from the group consisting of antibodies, antibody fragments, peptides, proteins, aptamers, molecularly imprinted polymers, biotin, streptavidin, his-tags, chelated metal ions such as Ni-NTA, oligonucleotides, or any combination thereof. In some embodiments, the sensor chip may further comprise at least a second sample reservoir. In some embodiments, the sensor chip may further comprise at least one reagent reservoir. In some embodiments, the sensor chip may further comprise at least one waste reservoir. In some embodiments, the sample reservoir further comprises a filtration membrane. In some embodiments, the sample reservoir is sealed. In some embodiments, the sample reservoir is sealed with a cap, a flexible membrane, or a septum. In some embodiments, the one or more reaction wells are sealed with an optically transparent material. In some embodiments, the optically transparent material is glass or a scatter-free polymer sheet. In some embodiments, the sensor chip further comprises at least one microfabricated pump. In some embodiments, the sensor chip further comprises at least one microfabricated valve. In some embodiments, a thickness of the sensor surface is about 15 nm to about 200 nm. In some embodiments, the sensor surface comprises two or more layers of material. In some embodiments, a thickness each layer is about 5 nm to about 100 nm. In some embodiments, each layer comprises metal, noble metal, polymer, ceramic, or glass. In some embodiments, a top layer has a primary binding component immobilized thereon, wherein the primary binding component is selected from the group consisting of antibodies, antibody fragments, peptides, proteins, aptamers, molecularly imprinted polymers, biotin, streptavidin, his-tags, chelated metal ions such as Ni-NTA, oligonucleotides, or any combination thereof, and wherein the top layer is a nanostructured, noble metal thin film. In some embodiments, the surface comprises a nanostructured, doped or self-doped semiconductor thin film. In some embodiments, the nanostructured, doped or self-doped semiconductor film is copper(I) sulphide (Cu₂-xS), a doped semiconductor-based oxide (including but not limited to aluminum-doped ZnO, gallium-doped ZnO, or indium-tin oxide) or a transition metal nitride such as nitrides of titanium (TiN), of tantalum (TaN), of hafnium (HfN) or of zirconium (ZnN). In some embodiments, the sensor surface comprises a nanostructured, metal thin film. In some embodiments, the nanostructured, metal thin film is a nanostructured, noble metal thin film. In some embodiments, the nanostructured, noble metal thin film is a nanostructured, gold thin film. In some embodiments, the reaction wells have a diameter of about 0.1 to about 5 mm. In some embodiments, the reaction wells have a cross-sectional area of less than about 20 μm². In some embodiments, the reaction wells have a depth of about 10 μm to about 2 mm. In some embodiments, each reaction well is configured to hold a volume of less than 25 μL. In some embodiments, the sample reservoir has a diameter of about 0.3 mm to about 10 mm. In some embodiments, the sample reservoir has a depth of about 0.03 mm to about 5 mm. In some embodiments, the fluid conduits have a substantially rectangular cross-section. In some embodiments, the fluid conduits have a width of about 0.1 mm to about 2.5 mm, and a depth of about 0.1 mm to about 2.5 mm. In some embodiments, the fluid conduits have a substantially circular cross-section. In some embodiments, the fluid conduits have a diameter of about 0.1 mm to about 2.5 mm. In some embodiments, the analyte-induced change in an optical property is a shift in the absorption maximum for light reflected from the sensor surface. In some embodiments, the analyte-induced change in an optical property is a change in the angle of reflection for light incident on the sensor surface at an oblique angle. In some embodiments, the analyte-induced change in an optical property is a change in a polarization of reflected light in respect to a polarization of light incident on the sensor surface.

Also disclosed herein are devices comprising: a) one or more sensor chips, wherein a sensor chip comprises: i) one or more sensor surfaces, wherein each sensor surface is capable of sustaining a localized surface plasmon resonance, and wherein each sensor surface is contained within a reaction well; ii) a sample reservoir configured to contain a sample comprising an analyte; and iii) one or more fluid conduits, wherein each fluid conduit connects the sample reservoir and one of the reaction wells; b) one or more light sources configured to illuminate the one or more sensor surfaces; and c) one or more detectors configured to detect an analyte-induced change in an optical property of light reflected or transmitted by the one or more sensor surfaces.

In some embodiments, the device further comprises one or more primary binding components immobilized on the one or more sensor surfaces, wherein the primary binding component is selected from the group consisting of antibodies, antibody fragments, peptides, proteins, aptamers, molecularly imprinted polymers, biotin, streptavidin, hist-tags, chelated metal ions such as Ni-NTA, oligonucleotides, or any combination thereof. In some embodiments, the device further comprises a housing that encloses the one or more sensor chips, one or more light sources, and one or more detectors. In some embodiments, the device further comprises a piston mechanism coupled to the sample reservoir to actuate flow of the sample through the one or more fluid conduits. In some embodiments, the device further comprises at least a second sample reservoir. In some embodiments, the device further comprises at least one reagent reservoir. In some embodiments, the device further comprises at least one waste reservoir. In some embodiments, the sample reservoir further comprises a filtration membrane. In some embodiments, the sample reservoir is sealed. In some embodiments, the sample reservoir is sealed with a cap, a flexible membrane, or a septum. In some embodiments, the one or more reaction wells is sealed with an optically transparent material. In some embodiments, the optically transparent material is glass or a scatter-free polymer sheet. In some embodiments, the device further comprises at least one pump. In some embodiments, the one or more sensor chips further comprise at least one microfabricated pump. In some embodiments, the device further comprises at least one valve. In some embodiments, the one or more sensor chips further comprise at least one microfabricated valve. In some embodiments, the sensor chip is a single-use disposable. In some embodiments, a thickness of the sensor surface is about 15 nm to about 200 nm. In some embodiments, the sensor surface comprises two or more layers of material. In some embodiments, a thickness of each layer is about 5 nm to about 100 nm. In some embodiments, each layer comprises metal, noble metal, polymer, ceramic, or glass. In some embodiments, a top layer has a primary binding component immobilized thereon, wherein the primary binding component is selected from the group consisting of antibodies, antibody fragments, peptides, proteins, aptamers, molecularly imprinted polymers, biotin, streptavidin, his-tags, chelated metal ions such as Ni-NTA, oligonucleotides, or any combination thereof, and wherein the top layer is a nanostructured, noble metal thin film. In some embodiments, the surface comprises a nanostructured, doped or self-doped semiconductor thin film. In some embodiments, the nanostructured, doped or self-doped semiconductor film is copper(I) sulphide (Cu₂-xS), a doped semiconductor-based oxide (including but not limited to aluminum-doped ZnO, gallium-doped ZnO, or indium-tin oxide) or a transition metal nitride such as nitrides of titanium (TiN), of tantalum (TaN), of hafnium (HfN) or of zirconium (ZnN). In some embodiments, sensor surface comprises a nanostructured, metal thin film. In some embodiments, the nanostructured, metal thin film is a nanostructured, noble metal thin film. In some embodiments, the nanostructured, noble metal thin film is a nanostructured, gold thin film. In some embodiments, the analyte-induced change in an optical property is a shift in the absorption maximum for light reflected from the sensor surface. In some embodiments, the analyte-induced change in an optical property is a change in the angle of reflection for light incident on the sensor surface at an oblique angle. In some embodiments, the analyte-induced change in an optical property is a change in a polarization of reflected light in respect to a polarization of light incident on the sensor surface. In some embodiments, the device is additionally configured to perform self-calibration functions. In some embodiments, the device further comprises a processor configured to perform data processing and storage functions. In some embodiments, the processor is a mobile phone or other smart device comprising a camera to which the device is connected via a USB cable. In some embodiments, the processor is a mobile phone or smart device comprising a camera to which the device is connected wirelessly. In some embodiments, the processor is further configured to transmit and receive data from the internet. In some embodiments, the device is configured as a benchtop device. In some embodiments, the device is configured as a hand-held device. In some embodiments, the device is configured as a wearable device. In some embodiments, the device further comprises microfabricated or nanofabricated needles, or another sample collection device, for drawing a blood sample. In some embodiments, the device is integrated with a consumer product.

Disclosed herein are devices comprising: a) one or more light sources configured to illuminate one or more sensor surfaces on a sensor chip; b) one or more detectors configured to detect an analyte-induced change in an optical property of light reflected or transmitted by the one or more sensor surfaces; and c) a piston configured to couple with a reservoir on the sensor chip to actuate flow of sample from the reservoir onto the one or more sensor surfaces.

Also disclosed herein are devices comprising: a) one or more sensor chips, wherein a sensor chip comprises: i) one or more sensor surfaces, wherein each sensor surface is capable of sustaining a localized surface plasmon resonance, and wherein each sensor surface is contained within a reaction well; ii) a sample reservoir configured to contain a sample comprising an analyte; and iii) one or more fluid conduits, wherein each fluid conduit connects the sample reservoir and one of the reaction wells; b) an optical system configured to capture images and detect an analyte induced change in an optical property of light reflected or transmitted by the one or more sensor surfaces; c) a processor for processing the images and determining a concentration of the analyte based on analysis of a series of two or more images, wherein the analyte-induced change is detected in one or more corresponding pixels in the series of two or more images near locations where analyte molecules are bound to the one or more sensor surfaces.

Disclosed herein are devices for detecting an analyte in a sample, the devices comprising: a) a substrate comprising one or more localized surface plasmon resonance (LSPR) sensors, wherein analytecortisol molecules are immobilized on a surface of the one or more LSPR sensors; and b) a cartridge, wherein the cartridge either partially or completely encloses the substrate, and wherein the surface(s) of the one or more LSPR sensors are accessible to addition of the sample.

In some embodiments, the device is configured to perform a competitive immunoassay for the detection and quantification of the analyte in the sample. In some embodiments, the analyte is selected from the group consisting of a peptide, a protein, an oligonucleotide, a lipid molecule, a carbohydrate molecule, a small organic molecule, a drug molecule, or any combination thereof. In some embodiments, the analyte is selected from the group consisting of glucose, cortisol, creatinine, lactate, C-reactive protein, alpha-fetoprotein, cardiac troponin I (cTnI), cardiac troponin T (cTNT), cardiac phosphocreatine kinase M and B (CK-MB), brain natriuretic peptide (BNP), or any combination thereof. In some embodiments, the analyte is cortisol. In some embodiments, the sample is diluted 1:1 by volume with a colloidal gold solution (OD=2) before addition to the one or more LSPR sensors. In some embodiments, the colloidal gold is coated with both an anti-analytecortisol antibody and alkaline phosphatase. In some embodiments, BCIP/NBT is used as a substrate for alkaline phosphatase. In some embodiments, the presence of the analyte in the sample is detected by means of a shift in the wavelength of light reflected from the one or more LSPR sensor surfaces. In some embodiments, a limit of detection for the competitive immunoassay performed in the device is better than about 1,000 pg/mL. In some embodiments, a limit of detection for the competitive immunoassay performed in the device is better than about 100 pg/mL. In some embodiments, a limit of detection for the competitive immunoassay performed in the device is better than about 10 pg/mL. In some embodiments, a limit of detection for the competitive immunoassay performed in the device is better than about 1 pg/mL. In some embodiments, the substrate comprises two or more LSPR sensors, and wherein at least one of the LSPR sensors is used to perform a control. In some embodiments, the sample is saliva. In some embodiments, the saliva is human saliva. In some embodiments, the sample is blood plasma or serum. In some embodiments, the cartridge comprises one or more reaction wells comprising the one or more LSPR sensors, and wherein the surface(s) of the one or more LSPR sensors are accessible to addition of the sample by pipetting the sample into the one or more reaction wells. In some embodiments, the cartridge comprises a sample reservoir and one or more reaction chambers comprising the one or more LSPR sensors, and the surface(s) of the one or more LSPR sensors are accessible to addition of the sample by flowing the sample from the sample reservoir to each of the one or more reaction chambers via interconnecting fluid channels. In some embodiments, the one or more reaction chambers are arranged in a hub-and-spoke pattern around a central sample reservoir. In some embodiments, the sample is caused to flow from the sample reservoir to each of the one or more reaction chambers via interconnecting fluid channels by exerting pressure on the sample reservoir using a mechanical piston. In some embodiments, the cartridge further comprises one or more valves for controlling the flow of sample or other fluids between the sample reservoir and the one or more reaction chambers. In some embodiments, the cartridge further comprises one or more reagent wells that are interconnected with the sample reservoir and the one or more reaction chambers via fluid channels. In some embodiments, the one or more reagent wells comprise pre-packaged assay reagents and/or controls.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety.

### BRIEF DESCRIPTION OF THE DRAWINGS

The novel features of the disclosed methods and devices are set forth with particularity in the appended claims. A better understanding of the features and advantages of the presently disclosed methods and devices will be obtained by reference to the following detailed description that sets forth illustrative embodiments, in which the principles of the novel designs are utilized, and the accompanying drawings of which:

FIG. 1 illustrates one embodiment of an ELISA-based LSPR assay in which an enzyme coupled to a secondary antibody (106) converts an enzyme substrate to an insoluble precipitate (108) that accumulates on the sensor surface when analyte (102) is captured by an immobilized capture antibody (104).

FIG. 2 illustrates one embodiment of a plasmon-plasmon coupling-based sandwich immunoassay LSPR assay in which a metallic nanoparticle or other particle capable of sustaining surface plasmons (201) is conjugated to a secondary antibody (203) induces strong coupling between nanoparticle surface plasmons and sensor surface plasmons when the metallic nanoparticle is brought into close proximity to the sensor surface.

FIGS. 3A and B illustrate spectroscopic detection of an analyte-induced shift in the extinction of white light reflected from an LSPR surface using the ELISA assay format. For each analyte concentration, the extinction of white light from the assay surface is measured before and after performing the enzymatic amplification step. At high analyte concentration (FIG. 3B), the before (306) and after (308) extinction spectra are clearly different, but below a certain analyte concentration, the difference in extinction spectra (310 and 312) becomes marginally small (FIG. 3A).

FIGS. 4A-C illustrate one embodiment of a digital LSPR detection technique in which an insoluble precipitate generated in an ELISA assay format, or a strong plasmon-plasmon coupling generated in a plasmon-plasmon coupling-based sandwich immunoassay, creates a local change in index of refraction of the LSPR-active surface that manifests itself as a local area with a distinct color change (indicated here as black spots). If the density of immobilized analyte is high, the entire LSPR surface is covered by black spots (FIG. 4C) and is detectable with either traditional spectroscopic or digital means. If however, the number of analyte molecules captured by the surface is small (at sample concentrations of ˜1 fg/ml or lower), the density of black spots is potentially very low (FIG. 4B) and would not be resolved by spectroscopic measurements.

FIG. 5 provides a conceptual illustration of one embodiment of a digital LSPR detection technique. Column A (analogue detection) shows simulated surfaces for an ELISA assay where a few, sparsely distributed ligands immobilized on the surface generate local color shifts that are essentially lost in the large areas of homogenous color. This analogue LSPR signal is dominated by the non-reacting areas that are identical for the three cases illustrated, i.e. where the analyte concentration increases going from case 1 to case 3. Column B (digital detection) illustrates a zoomed-in view of a section of the LSPR surface. At this higher magnification, the binding sites that produce a local color shift (darker spots) are clearly distinguishable from the homogenous background; this implementation of LSPR detection where the number of reaction spots are counted is called “Digital LSPR”. It provides for enhanced sensitivity in biological assays. In column C, histograms of the number of spots counted on randomly selected areas of the same LSPR surface are clearly able to distinguish between the three cases. Digital LSPR is capable of achieving lower detection limits than can be achieved by analogue LSPR.

FIG. 6 illustrates a reaction involving binding of ions or transfer of electrons on a LSPR surface and optical monitoring of the electrochemical processes taking place on the surface.

FIG. 7 illustrates the range of improvement in both assay time and limit-of-detection (LOD) that is achievable using the LSPR sensors and assay formats disclosed herein.

FIG. 8 illustrates one embodiment of a wafer comprising six LSPR sensor devices with integrated fluidic components that are removably attached to each other at their edges.

FIG. 9 illustrates one embodiment of a top cross sectional view of an LSPR sensor device with reaction wells or chambers, a reservoir, and fluid conduits.

FIG. 10 illustrates one embodiment of a side cross sectional view of an LSPR sensor device with reaction wells or chambers, a reservoir, and fluid conduits.

FIGS. 11A-C illustrate different optical detection configurations for use in the portable, optionally disposable, near-patient or point-of-care diagnostic LSPR devices and systems disclosed herein. FIG. 11A shows one non-limiting example of an optical detection scheme wherein the output from an optical detector, e.g. a photodiode, is converted to digital read-out. FIG. 11B shows one non-limiting example of an optical detection scheme wherein the output from the optical detector is read as an analogue signal. FIG. 11C shows one non-limiting example of an optical detection scheme for use in portable or benchtop readers wherein the output from an optical detector, e.g. a camera, CCD sensor, CMOS sensor, photodiode or photodiode array, etc., is converted to digital read-out.

FIGS. 12A-C illustrate sensor chip, assay device, and reader concepts for a compact, portable, benchtop diagnostics test system that utilizes the LSPR sensors and assay formats disclosed herein.

FIG. 13 illustrates a system concept for a hand-held point-of-care diagnostics test system in which a sensor card is read using an optical attachment that interfaces with a mobile phone.

FIGS. 14A-C illustrates part of the system concept for a hand-held point-of-care diagnostics test system in which a sensor card comprising one or more LSPR sensor chips is read using an optical attachment that interfaces with a mobile phone. In this concept, the mobile phone acts as the processor which acquires and processes the data from an LSPR sensor chip designed to perform a specific diagnostic test, e.g. cortisol test (FIGS. 14A and B), and displays the test result (FIG. 14C). In some embodiments, the mobile phone application is further configured to upload the test results to an internet cloud-based database and/or send a message to a designated family member or healthcare provider.

FIG. 15 shows a photograph of a wafer comprising a group of four detachable LSPR sensor devices that further comprise sample wells, reaction wells or chambers containing LSPR sensor surfaces, and interconnecting fluid channels.

FIG. 16 shows a photograph of a single LSPR sensor device after dicing the wafer illustrated in FIG. 15 into individual components.

FIG. 17 illustrates a wearable (watch-like) diagnostic test device concept that utilizes the LSPR sensors and assay formats disclosed herein.

FIGS. 18A and B further illustrate components of the wearable diagnostic test device concept that utilize the LSPR sensors and assay formats disclosed herein.

FIG. 19 illustrates the wearable diagnostic test device concept that utilizes the LSPR sensors and assay formats disclosed herein.

FIG. 20 shows examples of data for a cortisol competitive immunoassay performed using the LSPR sensors disclosed herein. Date obtained using two different sensors (indicated by the grey squares and black squares respectively) are shown.

### DETAILED DESCRIPTION OF THE INVENTION

Disclosed herein are LSPR sensor chips and point-of-care diagnostic devices or instruments that are potentially faster (e.g., assay times of approximately 15 minutes), more sensitive (up to four logarithms more sensitive; detection limits in the low femtomolar range), more robust, more economical, more reproducible, and smaller than currently available diagnostic devices and instruments. Additional advantages of the sensors and devices disclosed herein are the precise quantitation and manufacturing scalability of the sensors, which make them particularly well-suited for diagnostic instruments that are miniaturized, portable, and mobile for use at the point of care. Some embodiments described herein disclose diagnostic test instruments that are compact, portable, bench-top systems that are suitable for use in near-patient or point-of-care test settings. Some embodiments disclose hand-held diagnostic test devices that are suitable for use in near-patient or point-of-care test settings. Some embodiments described herein disclose diagnostic test devices that are wearable by a user. Thus, diagnostic testing that is equivalent in quality to that of the central labs may be provided wherever it is needed.

Overview of LSPR Sensor Technology:

Disclosed herein are methods and devices for highly sensitive detection of analytes in biological or chemical samples. The methods and devices disclosed exploit the phenomenon of localized surface plasmon resonance (LSPR) to optically detect binding of analyte molecules to a sensor surface. In some embodiments, detection may be based on direct measurement of the number of analyte molecules bound to the sensor surface. In some embodiments, detection may be based on an amplified signal that is proportional to the number of analyte molecules bound to the sensor surface. In some embodiments, detection is based on an analyte-induced change in a property of the sensor surface. In some embodiments, detection is based on high resolution imaging of the sensor surface that constitutes a paradigm shift in the way LSPR signals are collected and analyzed.

Surface plasmons are coherent, delocalized electron oscillations that exist at the interface between a negative and positive permittivity material, for example at a metal-dielectric interface such as a thin metal film exposed to an aqueous solution. Surface plasmon resonance occurs when the electron oscillations are induced by incident light, where the frequency of the incident photons matches the natural frequency of surface electrons oscillating against the restoring force exerted by positively charged nuclei distributed within the metal. Localized surface plasmon resonance occurs at the surface of small metallic nanoparticles or nanostructured surfaces upon excitation by light of the appropriate frequency. Localized surface plasmon resonance may also occur in doped or self-doped p-type semiconductor surfaces, such as copper(I) sulphide (Cu₂₋ₓS), a doped semiconductor-based oxide (including but not limited to aluminum-doped ZnO, gallium-doped ZnO, or indium-tin oxide) or a transition metal nitride such as nitrides of titanium (TiN), of tantalum (TaN), of hafnium (HfN) or of zirconium (ZnN).

LSPR sensors rely on the extreme sensitivity of the position of the surface plasmon absorption maximum to the local environment in the immediate vicinity of the interface. In particular, the signal transduction mechanism in LSPR biosensors is often associated with a change in the index of refraction (or dielectric constant) near an LSPR-active surface. If an LSPR sensor surface is placed in contact with a film or solution of index of refraction n₁, followed by deposition on the surface of a material having an index of refraction n₂, the wavelength of the plasmon absorption maximum shifts by a value Δλ. It is possible to link the plasmon shift to the change in index of refraction Δn=n₂−n₁ through the following relation:

Δλ=m*Δn[1−e^{(-2L/δ)}] (1)

where m is a constant representing the sensitivity of the sensor, L is the thickness of the deposited material with index of refraction n₂, and δ is the decay length of the evanescent plasmon field. In addition to monitoring the shift in absorption maximum, in some cases, the change in index of refraction (or dielectric constant) near the sensor surface may be detected by monitoring other optical properties, for example, changes in reflection angle of the incident light, changes in the intensity of transmitted light, changes in the polarization of light reflected from the surface, etc. The localization of surface plasmons in LSPR sensors derives from the use of metallic nanoparticles or nanostructured metallic surfaces. As will be described more fully below, there are a variety of approaches known to those of skill in the art for fabricating suitable sensor surfaces that are capable of sustaining a localized surface plasmon resonance. The optical properties of the surface, or of light transmitted or reflected by the surface, may then be monitored using any of a variety of light sources and detectors. In some embodiments of the disclosed methods and devices, a collimated white light beam provided by a simple LED source and appropriate optics is reflected from a nanostructured LSPR sensor surface, and the reflected light is monitored for a shift in absorption wavelength using a miniaturized spectrometer or other optical detector in order to detect analyte binding or analyte-dependent signal amplification events occurring on the sensor surface. In some embodiments, the sensor surface is imaged at high resolution, and local color shifts in the light reflected from the surface are monitored at the individual pixel level for extremely small (e.g. 3 pixel×3 pixel) regions of interest to detect analyte binding or analyte-dependent signal amplification events occurring on the sensor surface at analyte concentrations in the fg/ml range.

LSPR Sensors Coupled with ELISA Assays Formats:

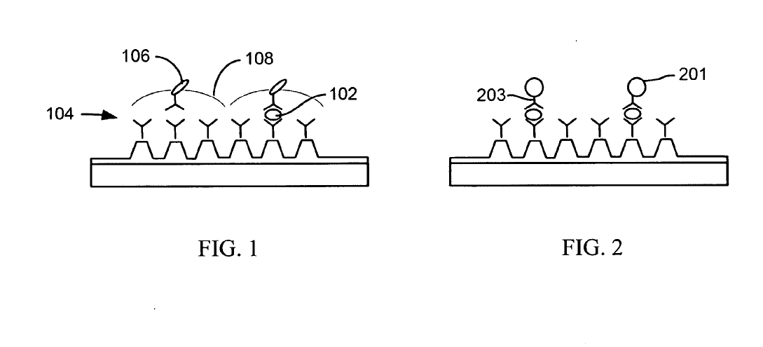

The ELISA assay format is a popular assay technique for the detection of analytes that relies on signal amplification to increase assay sensitivity. In some embodiments of the methods and devices disclosed herein, nanostructured LSPR sensor surfaces are combined with the immuno-precipitation ELISA assay format to achieve very low detection limits (e.g. in the fg/ml range). In this case, a primary antibody (104) directed towards the analyte (102) of interest is used to capture the analyte on the sensor surface, and a secondary antibody that is conjugated to a sensitivity enhancing label (106) binds to the immobilized analyte (FIG. 1). The sensitivity enhancing label may be, for example, an enzyme that catalyzes the conversion of a soluble reactant to an insoluble product that forms deposits on the sensor surface (108) near the location of the immobilized enzyme. The LSPR sensor then responds to the change of index of refraction (or dielectric constant) at the sensor surface which results from formation of the deposits. In some embodiments of the disclosed methods and devices, the enzyme used as a sensitivity enhancing label is alkaline phosphatase, which catalyzes the conversion of a mixture of 5-bromo-4-chloro-3′-indolyphosphate (BCIP) and nitro-blue tetrazolium (NBT) into a mixture of insoluble products. Other enzyme/substrate combinations are also possible, including but not limited to horse radish peroxidase (HRP)/tetramethylbenzidine (TMB), HRP/chloronaphtol (CN), HRP/diaminobenzidine (DAB), and HRP/CN-DAB. In general, any substrate for alkaline phosphatase or horse radish peroxidase may be used. This type of assay may be referred to herein as the “enzyme assay format.”

It is instructive to estimate the numerical values for the expected peak wavelength shift when a thin immuno-precipitate forms at the LSPR surface. For small deposits, equation 1 can be linearized and reduces to

$$Δ λ = m * Δ n \frac{2 L}{δ} \qquad ( 2 )$$

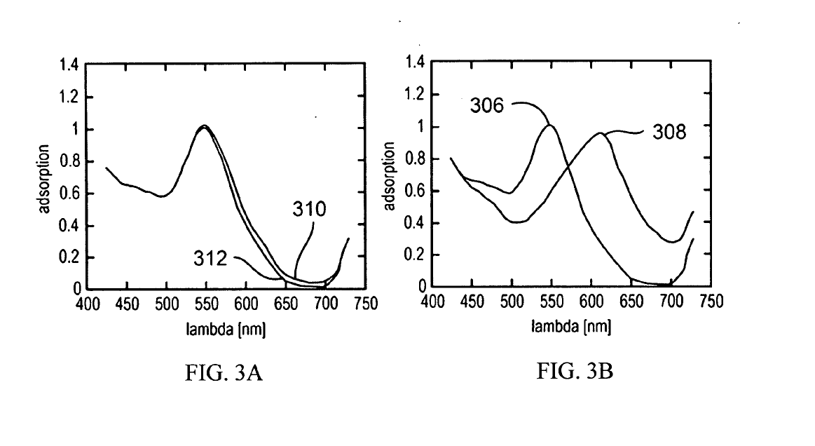

Using m/Δn=200, Δn=0.15 for deposition of BCIP/NBT with n₂˜1.48 and n₁=1.33, δ˜30 nm and L=5 nm, we obtain Δλ˜10 nm. Thus, a 5 nm deposit is predicted to generate a plasmon wavelength shift of ˜10 nm. In practice, wavelength shifts of up to 50-80 nm are observed. FIGS. 3A and B illustrate spectroscopic detection of an analyte-induced shift in the extinction of white light reflected from an LSPR surface using the ELISA assay format. For each analyte concentration, the extinction of white light from the assay surface is measured before and after performing the enzymatic amplification step. At high analyte concentration (FIG. 3B), the before (306) and after (308) extinction spectra are clearly different, but below a certain analyte concentration, the difference in extinction spectra (310 and 312) becomes marginally small (FIG. 3A). The implementation of immuno-precipitation ELISA assay formats on LSPR sensor surfaces has been found to improve the limit of detection for several assays by about 1 order of magnitude over that achieved using a conventional ELISA.

In addition to being sensitive to local refractive index changes as discussed above, there is another transduction mechanism for LSPR sensors that is able to generate large localized surface plasmon resonance shifts. The mechanism is based on plasmon-plasmon coupling. In this implementation, a plasmonic moiety, e.g. a particle capable of sustaining surface plasmons (201), is conjugated to the secondary antibody (203) as a sensitivity enhancing label (FIG. 2). The particle capable of sustaining surface plasmons may be noble metals, or their oxide counterparts, or noble metal core shell beads. Examples include colloidal gold and silver particles. Strong coupling between the particle plasmons and sensor surface plasmons occurs when the plasmonic particle anchors onto the plasmonic surface, and results in the measurements of exceedingly large plasmon shifts. As an example of resonance wavelength shifts that can be achieved by plasmon-plasmon coupling, consider a plasmonic particle such as a 40 nm gold colloid. When streptavidin binds to the surface of the 40 nm gold colloid, it produces approximately a 2 nm shift in the plasmon position of the gold colloid. In contrast, if streptavidin is attached to a 40 nm gold colloid and this biomolecule-gold colloid conjugate is brought into contact with a second plasmonic particle, the plasmon-plasmon coupling between colloidal particles produces an exceedingly large plasmon shift, potentially in excess of 70 nm. This phenomenon has been reported in the technical literature using pairs of colloidal particles in solution, and for other configurations with one colloidal particle in solution and a plasmonic partner on a surface. The type of assay wherein a secondary antibody is conjugated to a plasmonic particle and binds to an analyte molecule that has been captured on the LSPR surface by an immobilized primary antibody may be referred to herein as a “plasmon-plasmon coupling sandwich immunoassay” format. Both signal amplification mechanisms described above (i.e. the use of conjugated enzymes as sensitivity enhancement labels to catalyze reactions leading to local refractive index changes, and the use of conjugated metal nanoparticles to produce plasmon-plasmon coupling) result in plasmon shifts that can reach tens of nanometers in magnitude. The enhanced localized surface plasmon resonance shifts are associated with an enhanced limit of detection (LOD) in bioassays. In general, the LOD for ELISA assays performed on nanostructured LSPR surfaces are in the (sub-)pg/mL analyte range. For an average analyte of 60 kDa, these LOD correspond to approximately 10¹⁰ analyte molecules per milliliter of solution.

In some embodiments of the disclosed LSPR sensor chips and devices, signal amplification may be further enhanced by using a combination of both enzymatic amplification and plasmon-plasmon coupling. For example, multiple copies of an analyte-specific antibody and an enzyme molecule (e.g. alkaline phosphatase) may be coupled to colloidal gold particles, thereby resulting in both the formation of an insoluble precipitate on the sensor surface and plasmon-plasmon coupling between the gold particle and the sensor surface when analyte is present in a sample. Such an approach may dramatically increase the signal amplification achieved, thereby enabling faster assay times and/or lower limits of detection.

As described above, an analyte-induced change in the optical properties of the LSPR sensor surface may result from running an assay in either the ELISA assay format or the plasmon-plasmon coupling sandwich immunoassay format. Furthermore, these assays may be run as either a direct binding assay or a competitive binding assay. In a direct binding assay, primary binding components, e.g. capture antibodies, are immobilized on an LSPR surface and antigens are introduced with the sample to be tested. Secondary binding components, e.g. detection antibodies, may be added at the same time as the sample or in a subsequent step. When antigens present in the sample are captured by the immobilized captured antibodies, the labeled detection antibodies also become bound to the sensor surface and a change in an optical property of light reflected or transmitted by the surface occurs. An analyte-induced change may also result from running an assay with detection antibodies that are not conjugated to sensitivity enhancing labels. In this embodiment, an increase in mass occurs when the detection antibody binds to an analyte that has been captured on the LSPR surface by an immobilized primary antibody. The increase in mass results in a change in the index of refraction (or dielectric constant) at the sensor surface, which in turn leads to a change in an optical property of light reflected or transmitted by the surface. To further increase the change in mass, the detection antibodies may be conjugated with beads that increase mass, such as metal colloids, noble metal beads, magnetic beads, glass beads, or polymer beads.

Alternatively, LSPR sensor-based assays may be configured in a competitive binding assay format. In this approach, the presence of the antigen in a sample is detected by virtue of its ability to displace a labeled antigen present at a known concentration from binding to the capture antibody, and a detection antibody is not necessary. Increasing concentrations of the non-labeled antigen in the sample compete with the labeled antigen for binding to the capture antibodies on the LSPR sensor surface and prevent formation of the signal that would be observed in the absence of antigen in the sample.

Thus, analyte-induced changes in the optical properties of light transmitted by or reflected from LSPR sensor surfaces may be observed by configuring assays using any of a variety of assay formats and detection schemes, including but not limited to (1) direct binding assay formats, (2) competitive binding assay formats, (3) ELISA (enzyme-linked) assay formats, (4) plasmon-plasmon coupling sandwich immunoassay formats, (5) assays utilizing detection antibodies having no labels attached, and (6) assays utilizing detection antibodies that are conjugated with mass enhancing beads.

LSPR Sensors for Optical Readout of Electrochemical Reactions:

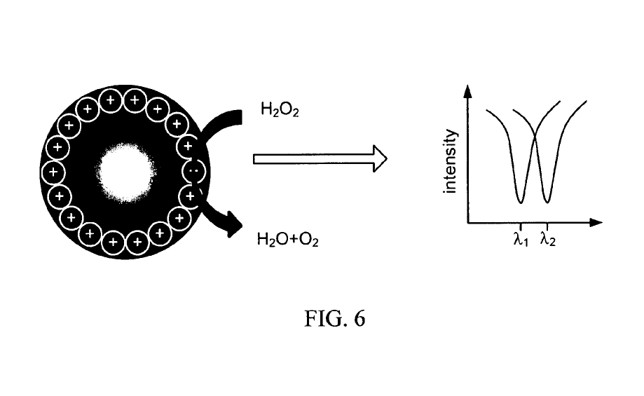

Also disclosed herein are methods and devices for enabling optical detection of electrochemical reactions taking place on the nanostructured LSPR surfaces. Electrochemical detection is widely used in diagnostics testing instruments, and particularly in point-of-care diagnostics testing devices. The localized surface plasmons sustained by nanostructured LSPR sensor surfaces render them very sensitive to reactions at the interface that involve binding of ions or transfer of electrons, for example, and enable optical monitoring of the electrochemical processes taking place on the surface (FIG. 6). Various detection modes are possible, including optical monitoring of chemical reactions taking place on unmodified sensor surfaces, optical monitoring of specific chemical reactions taking place on sensor surfaces that have been modified to construe reaction specificity, or monitoring of enzyme activity in a sample based on optical detection of electrochemical reactions at the sensor surface involving the reaction product for the enzymatically-catalyzed reaction.

Potential applications for these optical electrochemical sensor technologies include detection and measurement of small molecules, drugs, metal ions, gases, chemical compounds, small biological cofactors (e.g. molecules of less than 1000 daltons molecular weight), enzymes, and macromolecules. Electrochemical reactions may also be used to enhance the sensitivity of immunoassays performed on LSPR surfaces.

Potential advantages of these optical electrochemical sensor technologies include (i) enablement of rapid, simple, sensitive, and low-cost point-of-care diagnostics tests, (ii) faster time to test results, and (iii) the sensors are suitable for mass fabrication of miniaturized devices. Electrochemical detection is typically faster than colorimetric assays or ELISA-based assays employing colorimetric or fluorescence readout, for example, as the chemical reaction is typically monitored directly rather than waiting for accumulation of a colorimetric or fluorescent reaction product (which may take from several minutes to tens of minutes). Also, the assay process is typically simpler than that for an ELISA-based assay (i.e. requiring fewer steps, as there is typically no need for multiple binding steps involving the analyte and secondary antibodies, or multiple wash steps, for example). Electrochemical assay formats are also often less expensive than conventional ELISA-based colorimetric or fluorescent assays, due to the elimination of costly primary and secondary antibodies, labeling reagents, and other reactants, and may be easier to multiplex in that there is often no need to employ an analyte-specific surface (e.g. having an immobilized primary antibody that binds specifically to a single analyte). Often, the same surface and assay set up can be used to measure different compounds by simply changing the reagents used in the assay buffer. Finally, electrochemical-based assays have been demonstrated to exhibit higher sensitivity (i.e. lower LODs) in many cases than the corresponding colorimetric or ELISA-based assays.

ELISA-Based LSPR Sensors Coupled with Digital Imaging:

Also disclosed herein are methods and devices for further improving the sensitivity of ELISA-based LSPR biosensors. Most LSPR instruments using detection schemes based on measuring resonance peak shifts measure an analogue signal, i.e. the recorded signal is an average signal resulting from the binding of multiple analyte molecules on the surface. Individual binding events occur as random processes in time and space, and are not themselves directly detectable. Over time, the randomness gives rise to a well-defined average number of immobilized molecules on the surface. When the average number of immobilized molecules passes above the limit of detection, the instruments yield a positive reading. In reality, however, the binding of a single molecule to its ligand is a binary or digital process, i.e. it either binds or not. The ability to detect individual binding events therefore, may enable achievement of the ultimate assay sensitivity that can be reached.

The current disclosure provides methods and devices to further enhance the sensitivity (i.e. lower the limits of detection) for ELISA-type assays performed on nanostructured LSPR sensor surfaces by incorporating novel approaches for signal generation and analysis (i.e. digital LSPR). The approach may be applied to LSPR sensors coupled with ELISA assay formats exploiting either a conjugated enzyme (to catalyze formation of an insoluble precipitate on the sensor surface) or a conjugated metal nanoparticle (to induce plasmon-plasmon coupling) as sensitivity enhancing labels. In some embodiments, detection of analyte-induced optical properties (e.g. shifts in the plasmon resonance peak wavelength) utilizes white light illumination, an optical system capable of forming a magnified image of the LSPR sensor surface, a color or grey scale CCD camera to capture images, and an algorithm that measures the change in RGB or grey scale values for each pixel of the image.

Potential advantages of the disclosed methods and devices include both improved assay sensitivity and faster time to results. Note that the plasmon shift resulting from a change in refractive index (as predicted by Eq. 1) or from plasmon-plasmon coupling does not depend on the size of the LSPR sensing area; LSPR sensing areas as small as 20 nm have been successfully demonstrated. A difficulty with using such small sensing areas is that the number of photons collected is small. Therefore, analogue spectral analysis to measure peak shifts must use long integration times of much greater than 1 min. For the precision measurements required in a quantitative assay, the number of signal measurements required to reduce the intrinsic noise in the optical detection through signal averaging may bring the total signal collection time to greater than 10-100 min, since the signal to noise ratio scales as the number of signal sampling repeats, S/N˜\sqrt{repeats}.

A better detection scheme when a limited number of photons are reflected from a small sensing area is to image the sensing area at high resolution using a CCD or CMOS camera. In fact, local spectrometric shifts of 1-5 nm are equivalent to local color changes that can be captured in a color image and quantified through the change in RGB values for every pixel. Measuring the color for an individual image sensor pixel requires fewer photons than measuring the full spectrum of the light impinging on it. Therefore, color detection vs spectral detection provides the advantage of a fast sampling rate. Note however that both detection methods contain similar information. While spectroscopic detection is far superior for shifts in the 0-5 nm range for sensing areas on the order of mm² due to lower noise levels and the potentially large number of photons involved, imaging becomes a viable method for resonance peak shifts of greater than 5 nm for sensing area in the um² range and below where the photon count is limited.

FIGS. 1 and 2 illustrate two traditional ELISA assay formats on LSPR surfaces, where biomarkers (analytes) are detected through the use of the well-established sandwich assay format. As described above, the secondary antibody may be conjugated to a sensitivity enhancing label, e.g. an enzyme that catalyzes the conversion of a soluble substrate into an insoluble product (FIG. 1), thereby producing a change in the dielectric constant at the surface and thus a change in its reflectivity properties. The LSPR surface is illuminated with white light, and its reflectivity (or extinction) or transmission from the whole surface is measured. The reaction is quantified by the plasmon peak shift or any variation in the entire extinction spectrum. At low biomarker concentrations, only a few enzyme molecules become immobilized on the surface. Even though each enzyme will generate some insoluble product that deposits on the surface near the location of the enzyme reaction, the resulting spots will be few and far between. The extinction spectrum measured for the entire surface will not be significantly affected, and the assay will yield negative results.

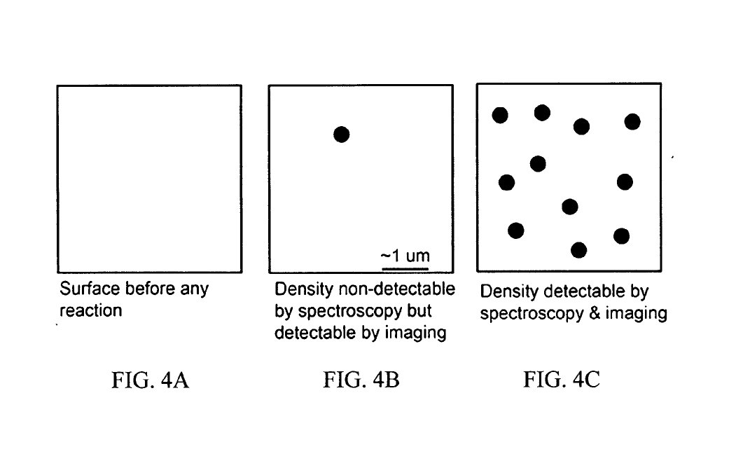

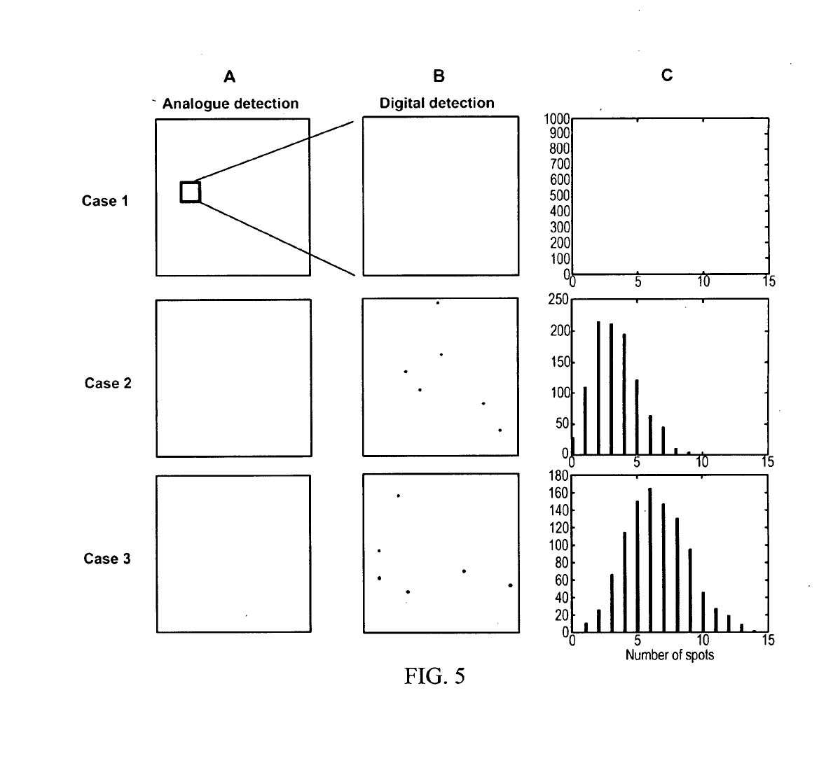

FIGS. 4A-C, and FIG. 5 illustrate the concept of digital LSPR for enhanced bioassay sensitivity. A color or grey scale image of a magnified area of the sensor surface is captured using a long-working distance objective. Depending on the magnification, the deposited precipitate spots corresponding to locations of enzyme activity are clearly distinguishable and can be counted. The precipitate-free areas and localized precipitate spots can be distinguished since the local dielectric constants are different, and thus their local extinction properties will differ. In particular, a red shifted extinction is expected for local areas of the sensor surface where precipitate has been deposited. The shift in extinction manifests itself as color difference between different areas of the surface that can be quantified by an analysis of their RGB values.

Currently, the extinction properties of LSPR nanostructures are typically observed using dark-field illumination. This type of illumination requires the objective to be in close contact with the surface. It is therefore not compatible with using a flow cell to dispense samples and rinse solutions for performing an ELISA assay on the LSPR surface. The novel approach disclosed herein bypasses this limitation by using a long-working distance objective.

FIGS. 4A-C & 5 illustrate the digital LSPR concept and its superiority over traditional LSPR assay formats in terms of limit of detection. For the three cases illustrated in FIG. 5, the number of marker (analyte) molecules immobilized on the sensor surface is below the level of detection for conventional spectral analysis. This is what is illustrated in the left-hand column, where an analogue signal (e.g., the color of the surface) does not allow differentiation between the three cases. By using a long working distance objective, however, it is possible to image the surface at a higher magnification. In the middle column for instance, a subsection of the entire sensor area is isolated and analyzed. In this case, faint spots of different color can be clearly discerned in cases 2 and 3, but not in case 1. If the number of spots for several randomly selected subsections of the entire image is counted and plotted in a histogram, there is a clear distinction between the three cases. The counting of spots (i.e. the locations of enzymatic reactions resulting from immobilization of the marker) constitutes a digital readout.

The digital strategy disclosed herein is possible through the marriage of ELISA or plasmon-plasmon coupled sandwich immunoassays to LSPR surfaces. If a generic (non-LSPR) surface was used, the deposition of the precipitate generated by a single enzyme molecule would not produce an optically detectable signal since the amount of precipitate deposited on the surface is too small to absorb light. In fact, the photo-absorption cross sections are relatively small for all dyes, thereby necessitating the deposition of thick layers of precipitate materials (>30-50 nm) over large areas (>>um²) to yield measurable absorption.

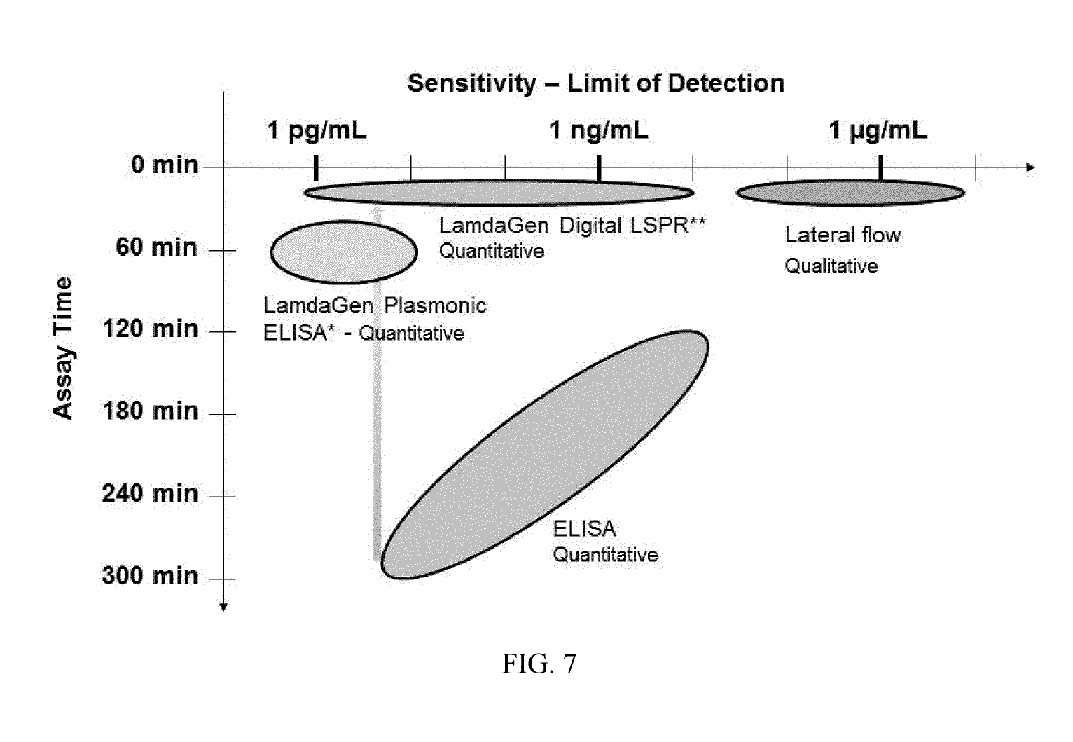

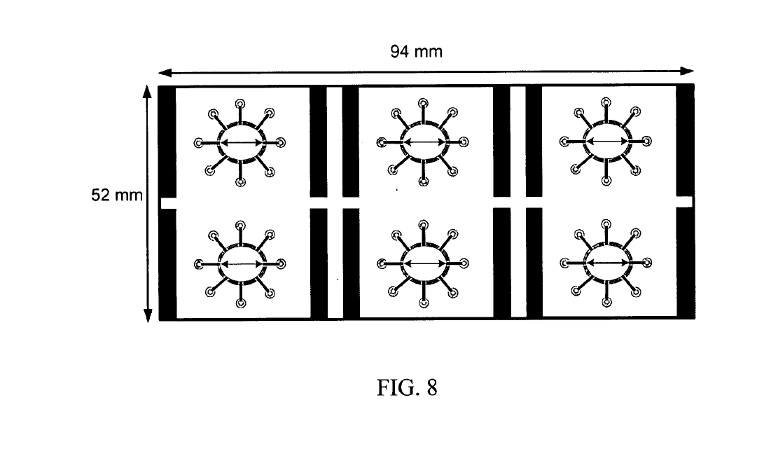

FIG. 7 illustrates the range of improvement in both assay time and limit-of-detection (LOD) that is achievable using the LSPR sensors and assay formats disclosed herein. Use of the LSPR sensor-based assay formats disclosed herein enable quantitative assay performance that achieves sensitivity (LODs of better than 1 pg/ml) exceeding that of conventional ELISA assays on timescales (e.g., several minutes) equivalent to those for conventional lateral flow assays.

Further optimization of assay parameters, e.g. optimization of the density of primary binding components on the sensor surface, sample incubation times, etc., and of detection parameters, e.g. the intensity and/or wavelength of light used to illuminate the sensor surface, the choice of low noise detector, etc., may push the achievable detection limits much lower than sub-fg/ml. In some embodiment, the limit of detection may be better than 1 mg/ml, 100 ug/ml, 10 ug/ml, 1 ug/ml, 100 ng/ml, 10 ng/ml, 1 ng/ml, 100 pg/ml, 10 pg/ml, 1 pg/ml, 100 fg/ml, 10 fg/ml, 1 fg/ml, or 0.1 fg/ml. Thus, the systems and methods disclosed herein may detect analytes present in a sample in an amount about or less than 100 mg/ml, 10 mg/ml, 1 mg/ml, 100 ug/ml, 10 ug/ml, 1 ug/ml, 100 ng/ml, 10 ng/ml, 1 ng/ml, 100 pg/ml, 10 pg/ml, 1 pg/ml, 100 fg/ml, 10 fg/ml, 1 fg/ml, or 0.1 fg/ml.

Nanostructured LSPR Sensor Surfaces:

A variety of methods may be used for fabricating nanostructured surfaces capable of sustaining localized surface plasmons, see for example, Takei, et al., U.S. Pat. No. 6,331,276, which is incorporated in its entirety herein. The components required to fabricate a nanostructured LSPR sensor may include substrates, metal layers or films, nanoparticles or nanostructures, and/or other dielectric or insulating materials. In some embodiments, the plasmon resonance properties of the LSPR sensor surface may be adjusted by manipulating the choice of materials, the number and ordering of layers, and the thickness of the layers used to fabricate the sensor.

Sensor Substrates:

Nanostructured LSPR sensors may be fabricated using a variety of materials, including, but not limited to, glass, fused-silica, silicon, ceramic, metal, or a polymer material. In some embodiments, it is desirable for the substrate material to be optically transparent so that the sensor surface may be illuminated from the back side. In other embodiments, the sensor surface is illuminated from the front side, and the transparency or opacity of the substrate material is not important. In some embodiments, it may be desirable to measure properties of light that is transmitted through the sensor surface. In some embodiments, it may be desirable to measure properties of light that is reflected from the sensor surface. For example, measuring properties of light reflected from the sensor surface may be superior than measuring light transmitted through the sensor surface in terms of plasmonic response to an analyte. In general, the substrates used for fabricating nanostructured LSPR sensors will have at least one flat surface, however, in some embodiments, the substrate may have a curved surface, e.g. a convex surface or a concave surface, or a surface of some other geometry.

Metal Layers or Films:

In general, nanostructured LSPR sensors may comprise one or more metal layers or metallic thin films. In some embodiments, there may be about 1, 2, 5, 10, 15, 20, or more metal layers. In some embodiments, the preferred metal for use in layers or films will be noble metals such as gold, silver, platinum, palladium, and the like. In some embodiments, non noble metals, e.g. copper, may be used. One advantage of using a noble metal is their ability to support surface plasmon activity due to the high mobility of conductance band electrons. For some noble metals, an additional advantage is their ability to resist chemical corrosion or oxidation. The metal layers or metallic thin films may comprise any mixture and/or any combination of the preferred metals mentioned herein. As an example, a film can comprise a “sandwich” of two layers of gold on the top and bottom and a layer of silver in between. As another example, a film can comprise a layer of gold metal, a layer of silver metal on top, and a layer of copper metal on top of the silver metal layer. The top layer may be nanostructured and made of a noble metal or metal oxides. In some embodiments, the top layer has antibodies immobilized on it for use in performing an assay. In addition, the other layers besides the top layer may also be made of noble metal or metal oxides. In some embodiments, the film contains only one layer. Metal layers or films may be fabricated by any of the techniques known to those of skill in the art, including, but not limited to, thermal deposition, electroplating, sputter coating, chemical vapor deposition, vacuum deposition, and the like. In some embodiments, the total thickness of the film is between about 5 nm to about 500 nm. In some embodiment, the total thickness of the metal film may be at least 5 nm, at least 10 nm, at least 25 nm, at least 50 nm, at least 75 nm, at least 100 nm, at least 200 nm, at least 300 nm, at least 400 nm, or at least 500 nm. In some embodiments, the total thickness of the metal film may be at most 500 nm, at most 400 nm, at most 300 nm, at most 200 nm, at most 100 nm, at most 75 nm, at most 50 nm, at most 25 nm, at most 10 nm, or at most 5 nm. Those of skill in the art will recognize that the total thickness of the metal film may have any value within this range, for example, about 95 nm. In some embodiments, each individual layer in the film has a thickness of about 5 nm to about 100 nm. The thicknesses of each individual layer may be different or may be the same. In some embodiments, the thickness of each individual layer may be at least 5 nm, at least 10 nm, at least 20 nm, at least 30 nm, at least 40 nm, at least 50 nm, at least 60 nm, at least 70 nm, at least 80 nm, at least 90 nm, or at least 100 nm. In some embodiments, the thickness of each individual layer may be at most 100 nm, at most 90 nm, at most 80 nm, at most 70 nm, at most 60 nm, at most 50 nm, at most 40 nm, at most 30 nm, at most 20 nm, at most 10 nm, or at most 5 nm. Those of skill in the art will recognize that the thickness of each individual layer may have any value with this range, for example, 28 nm.

Dielectric Layers:

In some embodiments, nanostructured LSPR sensors will include one or more layers of a dielectric (insulating) material. In some embodiments, there may be about 1, 2, 5, 10, 15, 20, or more dielectric layers. Any of a variety of materials may be used, including, but not limited to, glass, ceramic, or polymer materials such as polyimides, heteroaromatic polymers, poly(aryl ether)s, fluoropolymers, or hydrocarbon polymers lacking polar groups. Polymer layers or thin films may be fabricated by any of a variety of techniques known to those of skill in the art, including, but not limited to, solution casting and spin coating, chemical vapor deposition, plasma enhanced chemical vapor deposition, and the like. In some embodiments, the surface plasmon resonance properties of a nanostructured LSPR sensor, e.g. resonance wavelength, may be tuned by adjusting the thickness or dielectric constant of the material used to form an insulating layer between two metallic layers.

Particles:

In some embodiments, nanostructured or microstructured surfaces may be prepared by adsorbing or attaching particles, e.g. nanoparticles or fine particles, to substrate surface. Nanoparticles are particles of diameter ranging from 1 to 500 nanometers. Fine particles are particles of diameter ranging from 500 to 2,500 nanometers. The particles may be of any shape including, but not limited to, spherical, non-spherical cubic, cuboid, pyramidal, cylindrical, conical, oblong, star-shaped, in the form of short nanowires, hollow, porous, and the like. Any of a number of different particle types, or mixtures of particle types, may be used, including, but not limited to, metal particles, noble metal particles, metal-oxide particles, metal-alloy particles, metal-doped semi-conductor particles, nonmetal composite particles, polymer particles, gold or silver colloids, dielectric nanoparticles and microparticles, semiconductor nanoparticles, and hybrid structures such as core-shell nanoparticles, many of which are available commercially or can be prepared by any of a variety of methods known to those of skill in the art. Hybrid structures may be composed of different materials. For example, a core-shell nanoparticle may be comprised of a solid outer shell and a liquid inner core.

Coated Particle Surfaces:

In some embodiments, nanostructured LSPR surfaces are prepared by adsorbing or attaching non-metallic nanoparticles to a substrate surface and coating or partially-coating the attached particles with a thin metallic film to create a capped-particle surface, e.g. a gold-capped particle surface. The nanoparticles may be coated with one or more layers of the thin metallic film. For example, the nanoparticles may be coated with about 1, 2, 5, 10, 20 or more layers of the thin metallic film. In some embodiments, the preferred metal for use in the thin metallic film will be noble metals such as gold, silver, platinum, palladium, copper, and the like. The thin metallic film may comprise any mixture and/or any combination of the preferred metals mentioned herein. For example, the thin metallic film may comprise of one layer of gold, one layer of copper, and one layer of a mixture of silver and platinum. The coating may be of thickness between 5 nm and 200 nm. In some embodiments, the nanostructured surface may cover the entire substrate surface. In other embodiments, the nanostructured surface may cover only a portion of the substrate surface, and may be distributed across the substrate surface in a predefined pattern.

Alternative Nanostructured Surfaces:

In some embodiments, rather than utilizing nanoparticle adsorbed or attached to a surface to create nanostructured LSPR surfaces, the nanostructured surface may be fabricated using any of a variety of techniques known to those of skill in the art. Nanostructures such as cylindrical columns or pillars, rectangular columns or pillars, cylindrical or rectangular nanowells, and the like may be fabricated in a variety of substrate materials using techniques such as photolithography and wet chemical etching, reactive ion etching, or deep reactive ion etching, focused ion beam milling, application of heat to metal thin films to form islands, dip-pen nano lithography, and the like.

Dimensions and Patterns of Nanostructures on Surfaces:

The dimensions of the aforementioned nanostructures may range from a few nanometers to hundreds of nanometers. In some embodiments, the nanostructured surface may cover the entire substrate surface. In other embodiments, the nanostructured surface may cover only a portion of the substrate surface, and may be distributed across the substrate surface in a predefined pattern. The sensor surface may be capable of sustaining a localized surface plasmon resonance over all or portion of the sensor surface. The nanostructured surface may be of high or low density. To measure properties of light transmitted through a sensor surface, having a nanostructured surface of low density may be desired. To measure properties of light reflected from a sensor surface, having a nanostructured surface of high density may be desired. A surface having a high density of nanostructures may absorb and scatter light efficiently. In some embodiments, it may be desirable to measure properties of light that is transmitted through the sensor surface. In some embodiments, it may be desirable to measure properties of light that is reflected from the sensor surface. For example, measuring properties of light reflected from the sensor surface may be superior than measuring light transmitted through the sensor surface in terms of plasmonic response to an analyte.

Fabrication of the LSPR Active Surface:

LSPR active surfaces may be created from the components described above in a variety of ways and/or steps. As a non-limiting, illustrative example, a method of creating one type of LSPR active surface mentioned herein may comprise 1) the deposition of a thin film of Au in the range of 5-500 nm thick, 2) chemistry deposition of nanometer size silica or polymer particles (˜10 to 2500 nm in size) in a random, close-packed configuration, and 3) capping of the silica or polymer particles with one or more layers of Au (˜5 to 200 nm thick).

Assay Samples, Assay Analytes, and Assay Components:

As described above, a variety of assay (test) formats may be implemented using nanostructured LSPR sensors as a detector, including, but not limited to, sandwich immunoassays, enzyme-linked immunosorbent (ELISA) assays, electrochemical assays, and the like. Many of these assay formats require the use of affinity reagents (or binding components), e.g. antibodies, to confer binding specificity for the analyte of interest to the sensor surface.

Assay Samples:

Assays for the detection and quantitation of analytes in a variety of samples may be implemented using nanostructured LSPR sensors or devices that incorporate nanostructured LSPR sensors. Examples of samples include air, gas, water, soil, or industrial process stream samples, as well as biological samples such as tissue, cells, or any bodily fluid, such as blood, plasma, serum, sweat, tears, urine, or saliva from humans or animals, including from meat food products. In some embodiments, samples derived from animals or humans may be “patient samples”, and the results of the assay may be used in pathogen detection, disease diagnosis, or the making of treatment and healthcare decisions by a healthcare provider.

Assay Analytes:

Assays for the detection and quantitation of a variety of analytes (antigens, markers, biomarkers) may be implemented using nanostructured LSPR sensors, where the analyte may be present in small, moderate, or large quantities in a sample. The analyte may be any molecule of interest. An analyte may include, but is not limited to, an antigen, a peptide, a protein, an oligonucleotide, a DNA molecule, fragments of DNA, an RNA molecule, a ligand, a virus, a bacterium, environmental contaminants (e.g., contaminants in air, water, or soil samples), a cell, a pathogen, a lipid molecule, a carbohydrate molecule, a small organic molecule, a drug molecule, or an ion. The analyte may be any biomarker of interest in clinical diagnostic applications, e.g., glucose, cortisol, creatinine, lactate, C-reactive protein, alpha-fetoprotein, or cardiac marker tests (e.g., cardiac troponin I (cTnI), cardiac troponin T (cTNT), cardiac phosphocreatine kinase M and B (CK-MB), and brain natriuretic peptide (BNP)), as well an analyte of interest in non-human diagnostics (e.g. veterinary testing, animal feed stock testing), environmental testing (e.g. air, water, or soil testing), or industrial process monitoring sectors (e.g. bioreactor process monitoring).

Primary Binding Components:

Any of a variety of affinity reagents, affinity tags, or primary binding components may be used for recognition and binding of the target analyte with high specificity and high affinity, including, but not limited to antibodies (e.g., primary antibodies or capture antibodies), antibody fragments, peptides, proteins, aptamers, molecularly imprinted polymers, biotin, streptavidin, his-tags, chelated metal ions such as Ni-NTA, or DNA or RNA oligonucleotide probes, or any combination thereof. In some embodiments, one or more primary binding components may be pre-immobilized on the sensor surface prior to performing an assay using any of a variety of known surface immobilization techniques known to those of skill in the art, including but not limited to, non-specific adsorption; use of biotin-streptavidin linkages; use of silane chemistries to functionalize sensor substrate surfaces, followed by covalent chemical coupling to amine groups, carboxylate groups, etc.; use of poly-histidine tags and Ni-NTA chelators; and use of thiol-gold self-assembly techniques. In some embodiments, one or more primary binding components may be mixed with the sample prior to contacting the sensor surface with the sample, i.e. as part of the assay procedure.

Secondary Binding Components:

In some embodiments, a variety of affinity reagents, affinity tags, or secondary binding components may also be used to confer high specificity and enhanced sensitivity to the performance of the nanostructured LSPR sensor. In some embodiments, the secondary binding component may be conjugated to a sensitivity enhancing label to yet further increase the sensitivity of the assay. Examples of suitable secondary binding components for use in the methods and devices disclosed herein include, but are not limited to, antibodies (e.g., secondary antibodies or detection antibodies), antibody fragments, aptamers, molecularly imprinted polymer beads, biotin, streptavidin, his-tags, chelated metal ions such as Ni-NTA, oligonucleotide probes. Examples of sensitivity enhancing labels include (i) enzymes which catalyze the conversion of a non-detectable reactant to a detectable reaction product, e.g. an insoluble precipitate that forms deposit on the nanostructured LSPR sensor surface, and (ii) metallic nanoparticles or microparticles which are capable of inducing plasmon-plasmon coupling with the sensor surface. Examples of enzymes that may be suitable for use as sensitivity enhancing labels include, but are not limited to, alkaline phosphatase and horse radish peroxidase. Examples of reactants that may be suitable for enzymatic conversion to an insoluble precipitate that may form deposits on the sensor surface include, but are not limited to, 5-bromo-4-chloro-3′-indolyphosphate (BCIP) and nitro-blue tetrazolium (NBT), or mixtures thereof, which are converted to an insoluble precipitate by alkaline phosphatase.

Fluidic System Components:

The methods, devices, and systems of the present disclosure may utilize a fluidic system that is fully or partially integrated with one or more LSPR sensors. The fluidic system may be configured to deliver one or more samples and/or assay reagents to the one or more sensor surfaces. The fluidic system may comprise pumps or other fluid actuation mechanisms, valves, fluid channels or conduits, membranes, flow cells, reaction wells or chambers, and/or reservoirs with reagents necessary for carrying out the assay. In some embodiments, all or a portion of the fluidic system components may be integrated with the LSPR sensor to create LSPR chips or devices. In some embodiments, the LSPR chips or devices may be disposable or consumable devices. In some embodiments, all or a portion of the fluidic system components may reside in an external housing or instrument with which the LSPR sensor chip or device interfaces.

Fluid Actuation Mechanisms:

In some embodiments, the fluidic system may include one or more fluid actuation mechanisms. Examples of suitable fluid actuation mechanisms for use in the disclosed methods, devices, and systems include application of positive or negative pressure to one or more reaction wells, reaction chambers, or reagent reservoirs, electrokinetic forces, electrowetting forces, passive capillary action, and the like. Positive or negative pressure may be applied directly, e.g. through the use of mechanical actuators or pistons that are coupled to the reservoirs to actuate flow of the reagents from the reservoirs, through the fluid channels or conduits, and onto the sensor surface. In some embodiments, the mechanical actuators or pistons may exert force on a flexible membrane that is used to seal the reaction chambers or reservoirs. In some embodiments, positive or negative pressure may be applied indirectly, e.g. through the use of a pressurized gas lines or vacuum lines connected with one or more reservoirs. In some embodiment, pumps may be used to drive fluid flow. These may be pumps located in a housing or instrument with which an LSPR sensor chip interfaces, or in some embodiments they may be microfabricated pumps integrated with the sensor chip.

Fluid Channels:

In some embodiments, the fluid conduits may be have a substantially rectangular cross-section. In these embodiments, the fluid conduits may have a width of about 10 μm to about 5 mm, and a depth of about 10 μm to about 5 mm. In other embodiments, the fluid conduits may have a substantially circular cross-section. In these embodiments, the fluid conduits may have a diameter of between about 10 μm and about 5 mm.

Valves:

In some embodiments, the fluidic system may include one or more valves for switching fluid flow between reservoirs and channels. These may be valves located in a housing or instrument with which an LSPR sensor chip interfaces, or in some embodiments they may be microfabricated valves integrated with the sensor chip. Examples of suitable valves for use in the disclosed devices and instruments include solenoid valves, pneumatic valves, pinch valves, membrane valves, and the like.

Reaction Wells & Reaction Chambers:

The LSPR sensor chips disclosed herein may have one or more reaction wells or reaction chambers containing an LSPR sensor where an assay takes place. Some of the reaction wells or chambers may be control wells or chambers. The combination of fluid actuation mechanisms and fluid control components, e.g. pumps and valves, used in the fluidic system allows fluids from different reservoirs to be mixed and introduced into the reaction wells or chambers in the sequence required to perform a specific assay. The fluidic system may introduce the fluids from the different reservoirs in any order, either consecutively, or simultaneously. For example, for assays utilizing secondary antibody conjugates, after the sample is introduced into the reaction wells or chambers, a diluent from a diluent reservoir may be introduced in order to rinse the reaction wells or chambers. Afterwards, secondary antibody conjugates can be introduced into the reaction wells from the secondary antibody conjugate reservoir. Next, diluent can again be introduced in order to rinse the reaction wells or chambers. Next, a reagent, such as an enzyme substrate that is enzymatically converted to an insoluble precipitate, can be introduced into the reaction wells or chambers from the reagent reservoir. Thus, in some embodiments LSPR sensor chips may contain a sample reservoir, a diluent reservoir, a secondary conjugated antibody reservoir, a reagent reservoir, and a waste reservoir.

In another embodiment, instead of introducing the different fluids into the reaction wells or reaction chambers sequentially, the different component fluids may be pre-mixed and introduced in a single step. For example, the sample, diluent, and secondary antibodies (which can be un-conjugated, conjugated with an enzyme, conjugated with a mass-enhancing particle, or conjugated with a plasmonic moiety), may be pre-mixed in a reservoir. Next, the pre-mixed fluid containing the diluted sample and secondary antibodies may be introduced into the reaction wells or chambers. In these embodiments, the LSPR sensor chips may contain a reservoir containing diluent and secondary antibodies, which can be mixed with the sample when the sample is introduced into that reservoir. Further, the LSPR sensor chips may also contain additional diluent reservoirs for rinsing, as well as waste reservoirs. In some embodiments, single step assays are performed by mixing the sample with a secondary binding component, e.g. an Ag/Au nanoparticle-conjugated antibodies, either before pipetting into the LSPR sensor device, or within a reaction well of the LSPR sensor device, and the presence of the analyte is detected directly without the need for separation or rinse steps.

The diameter of the reaction wells or chambers may range from about 100 μm to about 5 mm in diameter. The reaction wells or chambers need not be circular in shape. In some embodiments, the cross-sectional area of the reaction wells or chambers may range from about 20 μm² to about 25 mm². In some embodiments, the depth of the reaction wells or chambers may range from about 10 μm to about 10 mm deep. For example, the depth of a reaction well or chamber may be around 35 μm. In some embodiments, the volume of the reaction wells may range from 100 nanoliters to 3 milliliters. In some embodiments, the reaction wells may be configured to hold a volume of less than 25 μL.

In some embodiments, the LSPR sensor chip may have a plurality of reaction wells or chamber, wherein each contains a sensor. In some embodiments, the LSPR sensor chips may have a single reaction well or chamber containing an array of sensors. The LSPR sensors may be multi-paneled or multiplexed, such that a different type of assay may be run in each reaction well or chamber. Thus, different reaction wells may contain different antibodies, DNA for running DNA assays, RNA, bacteria, and so forth that are immobilized in the reaction wells. In some embodiments, the LSPR sensor may have multiple primary antibodies or other primary binding components immobilized on a single sensor surface. In some embodiments, some of the reaction wells may be control wells.

Reservoirs:

In some embodiments, the LSPR sensor chip may include one or more sample or reagent reservoirs. The reagents in the reservoirs may be introduced onto the sensor surface through the fluid channels. The reservoirs may contain samples, reagents, diluents, un-conjugated antibodies, antibodies conjugated with enzymes, antibodies conjugated with mass-enhancing beads, antibodies conjugated with a plasmonic moieties, assay controls, and/or waste products resulting from running an assay.

For example, for assays that are run sequentially, the LSPR sensor chip may contain one or more reservoirs for storing diluent, one or more reservoirs for storing antibodies (which may be un-conjugated, conjugated with enzymes, conjugated with mass-enhancing beads, or conjugated with plasmonic moieties), and one or more reservoirs for storing buffers or other assay reagents. Further, the LSPR sensor chip may also contain one or more waste reservoirs.

In other embodiments, the LSPR sensor chip may contain reservoirs which contain diluent and secondary antibodies (which may be un-conjugated, conjugated with enzymes, conjugated with mass-enhancing particles, or conjugated with plasmonic moieties), in the same reservoir. When the sample is introduced into this reservoir, the sample may be mixed with the diluent and the secondary antibodies. The entire mixture may then flow into the reaction wells where the assay takes place. Reagents may be stored in the LSPR sensor chips and devices in a variety of formats, including but not limited to, in solution, as freeze-dried (lyophilized) reagents, in the presence of stabilizing agents, e.g. polymers, etc., or in any combination thereof. In some embodiment, LSPR sensor chips may comprise fluid channels containing lyophilized assay reagents such that the reagents are solubilized when sample and/or assay buffers are added to the device. The LSPR sensor chip may also contain additional diluent reservoirs for washing, as well as waste reservoirs.

In some embodiments, the reservoirs may have a diameter of about 0.3 mm to about 10 mm, and a depth of about 0.03 mm to about 5 mm, or may have dimensions such that the volume is between 1 nL and 3 mL.

In some embodiments, the diameter of the reaction chambers or reservoirs may be at least 0.1 mm, at least 0.2 mm, at least 0.3 mm, at least 0.4 mm, at least 0.5 mm, at least 1 mm, at least 1.5 mm, at least 2 mm, at least 3 mm, at least 4 mm, at least 5 mm, at least 6 mm, at least 7 mm, at least 8 mm, at least 9 mm, or at least 10 mm. In some embodiments, the diameter of the reaction chambers or reservoirs may be at most 10 mm, at most 9 mm, at most 8 mm, at most 7 mm, at most 6 mm, at most 5 mm, at most 4 mm, at most 3 mm, at most 2 mm, at most 1.5 mm, at most 1 mm, at most 0.5 mm, at most 0.4 mm, at most 0.3 mm, at most 0.2 mm, or at most 0.1 mm. Those of skill in the art will recognize that the diameter of the reaction chambers or reservoirs may have any value within this range, e.g. about 2.4 mm. Similarly, in some embodiment, the depth of the reaction chambers or reservoirs may be at least 0.01 mm, at least 0.02 mm, at least 0.03 mm, at least 0.04 mm, at least 0.05 mm, at least 0.1 mm, at least 2 mm, at least 3 mm, at least 4 mm, or at least 5 mm. In some embodiments, the depth of the reaction chambers or reservoirs may be at most 5 mm, at most 4 mm, at most 3 mm, at most 2 mm, at most 1 mm, at most 0.5 mm, at most 0.4 mm, at most 0.3 mm, at most 0.3 mm, at most 0.2 mm, or at most 0.1 mm. The depth of the reaction chambers or reservoir may have any value with this range, e.g., about 0.55 mm. In some embodiments, the volume of the reaction chambers or reservoirs may be at least 1 nL, at least 5 nL, at least 10 nL, at least 25 nL, at least 50 nL, at least 100 nL, at least 200 nL, at least 300 nL, at least 400 nL, at least 500 nL, at least 1 mL, at least 1.5 mL, at least 2 mL, or at least 3 mL. In some embodiments, the volume of the reaction chambers or reservoirs may be at most 3 mL, at most 2 mL, at most 1.5 mL, at most 1 mL, at most 500 nL, at most 400 nL, at most 300 nL, at most 200 nL, at most 100 nL, at most 50 nL, at most 25 nL, at most 10 nL, at most 5 nL, or at most 1 nL. Those of skill in the art will recognize that the volume of the reaction chambers or reservoirs may have any value with this range, e.g., about 550 nL.

Membranes:

In some embodiments, there may be a membrane that serves as a filter placed on top of the reaction wells or sample reservoirs. In some embodiments, the sample to be assayed may be deposited onto the LSPR sensor surface by depositing the sample directly onto a membrane filter that covers the reaction well. The membrane filter may be designed to filter out unwanted particles according to size. For example, the filter may contain appropriately sized pores that only allow smaller sized particles to filter through to the reaction wells. Unwanted particles may include cells, salts crystals, insoluble precipitates, or other particulates which may interfere with the assay or clog the fluid conduits. A sample may contain one or more molecules of interest which may be separated by the membrane. Thus, different types of molecules may filter through to different reaction wells, and membranes of different porosity or different selectivity may enable the concurrent analysis of more than one analyte in a sample. In some embodiments, the sample is introduced by depositing it into a reservoir instead of or in addition to into a reaction well. The LSPR sensor may contain one or more reservoirs especially adapted to receive samples. The sample reservoirs may or may not include membranes placed on top of the reservoirs depending on whether or not filtering is desired. In some embodiments, filtration may be achieved by applying pressure on the sample with, for example, a piston. When the piston applies pressure on the sample, the smaller particles may be forced through the filtration membrane while the larger particles do not pass through the filtration membrane. Filtration may also be achieved without applying positive mechanical pressure. For example, filtration may be achieved by gravitational forces or through negative pressure applied from the side of the filtration membrane opposite where the sample lies.

Fabrication Materials, Techniques, and Dimensions:

In general, the reaction wells, reaction chambers, sample and reagent reservoirs, and fluid conduits may be fabricated using any of a variety of materials, including, but not limited to glass, fused-silica, silicon, polycarbonate, polymethylmethacrylate, cyclic olefin copolymer (COC) or cyclic olefin polymer (COP), polydimethylsiloxane (PDMS), or other elastomeric materials. Suitable fabrication techniques i(depending on the choice of material) include, but are not limited to, CNC machining, photolithography and etching, laser photoablation, injection molding, hot embossing, die cutting, and the like.

The size and shape of the fluid conduits, as well as the pressure applied to the one or more reaction wells, reaction chambers, or reservoirs, may be designed such that flow into the reaction wells is laminar. In some embodiments, the length of the fluid conduits may range from about 1 mm to about 100 mm. In some embodiments, the fluid conduits may be have a substantially rectangular cross-section. In these embodiments, the fluid conduits may have a width of about 10 μm to about 2.5 mm, and a depth of about 10 μm to about 2.5 mm. In other embodiments, the fluid conduits may have a substantially circular cross-section. In these embodiments, the fluid conduits may have a diameter of between about 10 μm and about 2.5 mm.

Optical System Components:

The methods, devices, and systems described herein may make use of LSPR sensor surfaces coupled with optical systems. An optical system may comprise one or more light sources, one or more objective lenses, additional lenses, apertures, mirrors, filters, beam splitters, prisms, one or more detectors (e.g., photodiodes, photodiode arrays, photomultiplier tubes, CCD cameras, CMOS sensors, etc.), and/or translation stages that may be scanned or maintained in a fixed position with respect to a detector, as well as microprocessors, computers, computer readable media, and the like.

In some embodiments, optical instruments may be designed to illuminate the LSPR sensor surfaces from the back side, in which case it is desirable for the substrate material to be optically transparent. In other embodiments, the sensor surface may be illuminated from the front side, and the transparency or opacity of the sensor substrate material is not important. In some embodiments, it may be desirable to measure properties of light that is transmitted through the sensor surface. In many embodiments, it is desirable to measure properties of light that is reflected from the sensor surface. For example, measuring the properties of light reflected from the sensor surface may be superior to measuring light transmitted through the sensor surface in terms of the ability to monitor the plasmonic response to an analyte. Any of a variety of physical properties of the light transmitted by or reflected from the LSPR sensor surface may be measured, e.g. spectra and/or spectral shifts, intensity, polarization, or angle of reflection.

In some embodiments, one or more microfabricated optical components, e.g. light sources, lenses, band-pass filters, waveguides, and/or detectors, may be directly integrated with LSPR sensors devices using manufacturing techniques adopted from the microelectromechanical systems (MEMS) industry.

Light Sources:

The light source may be an LED, laser, laser diode, halogen source, or any other suitable light source. The light source may direct light at the sensor surface before, during, and/or after an assay reaction takes place on the sensor surface. In some embodiments, the light source may be shuttered so that the sensor surface may be illuminated at selected times. In some embodiments, the light source may be pulsed at a pre-specified frequency so that signal-to-noise ratios for detection of the transmitted or reflected light may be improved through frequency-dependent amplification or boxcar integration techniques. The light source may direct light from the substrate side or from the sensor surface side. Often the light source may be a white light source, but in some embodiments of the disclosed methods, devices, and systems, monochromatic, narrowband, or broadband light may be used.

The light source may be placed such that light is generally incident on the LSPR surface at 90 degrees. Similarly, a detector may be placed such that it detects light that is reflected from the surface at 90 degrees. The light source may be placed such that light is generally incident on the LSPR surface at an oblique angle. The light may be configured to be narrow and collimated. Similarly, the detector may be placed such that it detects the reflected light form the surface at an oblique angle. The light source may be directed through an optical channel or an optical fiber. The optical channel or optical fiber may then be positioned so that light exits the optical channel or optical fiber and is incident on the LSPR surface at the desired angle.

Detectors:

The one or more detector(s) may be a photodiode, avalanche photodiode, photomultiplier tube, an image sensor, any other form of suitable light detector, or any combination thereof. In some embodiments, one or more detectors may be used to detect light transmitted by or reflected light from the LSPR sensor surface before, during, and/or after the assay is performed, thereby enabling the collection of endpoint assay determinations and/or kinetic assay data. A detector may detect a shift in the optical absorption peak before and after the plasmon-plasmon coupling or the ELISA reaction. The detector may detect any optical property of light, such as absorption peak, angle of reflected light, and polarization properties of light. In some embodiments, the detector may detect white light reflected from or transmitted by the sensor surface. In some embodiments, the detector may detect the transmitted or reflected light after it has passed through a prism, one or more bandpass filters, or a monochromator. The detector may comprise an image sensor. An image sensor may be a CCD sensor, CMOS sensor, or NMOS sensor. The image sensor may capture a series of images of the sensor surface. The series of images may be greyscale images. The series of images may be RGB images. The series of images may comprise image frames that correspond to images captured before, during, and after an assay is completed with the analyte. The series of images described herein may be of sufficient detail such that a change due to an analyte can be detected over the series of time lapse images. The series of images may comprise about or more than 1000 images, 500 images, 400 images, 300 images, 200 images, 100 images, 50 images, or 10 images. The image sensor may capture the series of image frames at a predefined capture rate. The inverse of the capture rate may be 1 millisecond per frame, 2 milliseconds per frame, 5 milliseconds per frame, 10 milliseconds per frame, 20 milliseconds per frame, or 50 milliseconds per frame. Image sensors may vary in terms of pixel size and pixel count. The image resolution may depend on the pixel size and pixel count. Image sensors may have a pixel count of about or more than 0.5 mega pixels, 1 mega pixels, 4 mega pixels, 10 mega pixels, 20 mega pixels, 50 mega pixels, 80 mega pixels, 100 mega pixels, 200 mega pixels, 500 mega pixels, or 1000 mega pixels. The pixel size corresponding to the image sensor may be about or less than 5 microns, 3.5 microns, 2 microns, 1 micron, 0.5 microns, or 0.1 micron.

Illumination and Collection Optics:

As indicated above, optical devices and instruments suitable for use with the LSPR sensor surfaces described herein will typically also include other optical components, e.g. lenses, mirrors, filters, beam-splitters, prisms, polarizers, optical fibers, and the like, for assembly of illumination and collection optical subsystems. In some embodiments, an epi-illumination design may be used such that a single objective lens acts to both deliver illumination light to the LSPR sensor surface and collect reflected light from the LSPR sensor surface. The objective lens (and collection optical sub-system) may provide a magnification of the sensor surface. The objective lens may have long working distance (e.g., 2-5 mm) to provide enough clearance to accommodate fluidic systems designed to deliver samples and assay reagents to the sensor surface. In some embodiments, the objective lens may be optimized for near-field imaging. The optical system may provide an overall magnification that is about 5×, 10×, 20×, 50×, 100×, 200×, or higher. The magnification of the optical system enables each pixel of the image frame to correspond to a surface area that is much smaller than the pixel size. For example, an image sensor with a pixel size of 5 microns capturing an image under a 10× objective will produce an image with a pixel that corresponds to a sensor surface of 0.25 μm². This magnification may enable local areas on the LSPR surface corresponding to enzyme activity or plasmon-plasmon coupling to be clearly distinguishable and counted.

Detection of Plasmon Peak Shifts:

The LSPR sensors and devices of the present disclosure may utilize algorithms for detecting plasmon peak shifts with high sensitivity. In general, binding of analytes or secondary antibodies to the sensor surface will induce a red-shift in the plasmon absorption maximum. However, in some embodiments, for example, an enzyme activity assay that monitors a protease that cleaves an immobilized substrate and removes material from the sensor surface, a blue-shift in the plasmon absorption maximum may be observed. In some embodiments of the disclosed methods, devices, and systems, plasmon peak shifts may be detected and/or quantified by monitoring reflected or transmitted light intensity at a single wavelength, e.g. at 620 nm. If the single wavelength is chosen to be on the blue side of the known plasmon absorption maximum, then an analyte-induced red shift will cause a decrease in intensity at the chosen wavelength. If the single wavelength is chosen to be on the red side of the known plasmon absorption maximum, then an analyte-induced red-shift will cause an increase in intensity at the chosen wavelength.

In some embodiments, plasmon peak shifts may be detected and/or quantified by monitoring reflected or transmitted light at two or more wavelengths. If the two or more wavelengths are chosen to flank the known plasmon absorption maximum, then monitoring the ratio of intensities at the two wavelengths, e.g. I_{red}/I_{blue}, where I_{red} is the intensity at a wavelength on the red side of the plasmon absorption maximum and I_{blue} is the intensity at a wavelength on the blue side of the plasmon absorption maximum, may provide a very sensitive means for detecting analyte-induced red shifts.

In some embodiments of the present disclosure, more advanced algorithms may be utilized to detect and/or quantify analyte-induced shifts in plasmon absorption maximum for improved signal-to-noise ratios and enhanced assay sensitivity. For example, polynomial fitting of the shape of the plasmon absorption curves before and after exposure of the sensor surface to an analyte may be followed up by various mathematical operations such as calculation of difference spectra, calculation of moments, or calculation of centroids, and the like. Additional examples of algorithms that may be usefully employed include, but are not limited to, signal averaging algorithms, signal smoothing algorithms (e.g. the Savitsky-Golay algorithm), pattern mining algorithms that delineate areas of the sensor surface that exhibit response to contact by an analyte, and the like. The pattern mining algorithms may manipulate changes in RGB or greyscale values to determine specific patterns on an image (e.g., determining areas of an LSPR sensor surface for which image pixels have undergone a change in red pixel value within a certain defined range). In some embodiments, the algorithm may determine a concentration of the analyte in a sample. Several known concentrations of the analyte and a corresponding signal that they generate may be measured and used for the generation of a calibration curve. An analyte may be detected as described herein, and the signal measured may then be compared to the calibration curve to determine a concentration of the analyte in a sample. Algorithms may be stored in a computer readable medium. The computer readable medium may be any medium capable of storing data in a format that may be read or processed by a device (e.g., compact disc, floppy disk, USB flash drive, hard disk drive, etc).

Diagnostic Devices & Applications:

Disclosed herein are devices and systems for use in diagnostic testing applications that incorporate LSPR sensor chips. In some embodiments, a bodily fluid (e.g., blood, plasma, serum, sweat, tears, urine, saliva, etc.) or other fluid (e.g., contaminated water, blood from meat food products, etc.), may be deposited onto an LSPR sensor chip for the purpose of performing an assay to detect and/or quantify the presence of one or more analytes contained therein. In some embodiments, the disclosed devices and systems may be capable of running assays using very small sample volumes (e.g., 25 μL or less). In some embodiments, the sample volumes required to perform an assay may be at least 0.1 μl, at least 0.5 μl, at least 1 μl, at least 2 μl, at least 3 μl, at least 4 μl, at least 5 μl, at least 10 μl, at least 15μ, at least 20 μl, or at least 25 μl. In some embodiments, the sample volumes required to perform an assay may be at most 25 μl, at most 20 μl, at most 15 μl, at most 10 μl, at most 5 μl, at most 4 μl, at most 3 μl, at most 2 μl, at most 1 μl, at most 0.5 μl, or at most 0.1 μl. Those of skill in the art will recognize that the sample volumes required may have any value within this range, for example, about 7.5 μl.

In some embodiments, the LSPR chip may be a reusable component of a diagnostic testing device or system. In many embodiments, the LSPR sensor chip may be a disposable device suitable for one-time use that may be discarded after a sample is deposited onto the sensor chip and analyzed. In some embodiments, the LSPR sensor chip may be interfaced with a microfluidics chip, or incorporated into a cartridge, a cassette, a lateral flow device, a package, or any other form of housing device, which may contain additional components for carrying out the assay. In some embodiments, the sample is collected and deposited onto the LSPR sensor chip after the LSPR sensor chip is interfaced or packaged with the housing device. For example, the housing device may contain components for collecting and depositing a sample onto the LSPR sensor chip.

As mentioned, the LSPR sensor chip may be integrated with microfluidics or packaged in a cartridge for carrying out assays. For example, as described above, the sensor device or cartridge may contain pumps, valves, and reservoirs with reagents necessary for carrying out the assay. The LSPR sensor device may also contain reaction wells or chambers where the assays take place. The reagents in the reservoirs may be introduced into the reaction wells or chambers through fluid conduits incorporated into the LSPR sensor device. In some embodiments, application of positive or negative pressure to one or more reservoirs on the sensor device may provide a means to actuate fluid flow. For example, pistons may be coupled to the reservoirs to actuate flow of the reagents from the reservoirs, through the conduits and into the reaction chambers. Flow may be actuated by an active mechanism, such as pumping or suction. Flow may also be actuated by passive capillary action. An instrument system or reader with which the sensor device interfaces may contain a white light source, a detector, and other components for carrying out and analyzing the results of assays. The instrument system or reader may also contain components (e.g., pumps and valves) to actuate and control fluid flow. The housing device may be reusable.

After the assay takes place in the reaction wells or chamber of the LSPR sensor device, a detector may be used to detect changes in an optical property of the LSPR sensor surface that resulted from the assay. A processor in the instrument or reader may be used to analyze the results. The results may then be displayed to the user or transmitted to a health care professional.

The near-patient testing and point-of-care diagnostic devices and instruments disclosed herein have a variety of in-vitro diagnostic applications. For example, a user may deposit a blood sample onto the LSPR sensor chip, and the sensor device or instrument may display information about the amount of Troponin I, which is a biomarker used in the early diagnosis of myocardial infarction. The diagnostic devices and instruments disclosed herein may also assay for C-reactive protein, another cardiovascular biomarker. The LSPR sensor chip and the housing device may be used to display quantitative data for a variety of other analytes as well, including but not limited to those which serve as markers for infectious disease (e.g., influenza A, influenza B, respiratory syncytial virus, or other pathogens), food safety (e.g., O157:H7 E. coli or other food-borne pathogens), metabolic disease, neurodegenerative disease, vector-borne disease, drugs of abuse (e.g., tetrahydrocannabinol, phencyclidine), diabetes (e.g., insulin resistance, glucose monitoring), cancer biomarkers (e.g., alpha-fetoprotein for liver cancer, thyroid stimulating hormone for thyroid cancer, E6 oncoprotein for cervical cancer), endocrine markers (e.g. cortisol), veterinary disease (e.g., Johne's disease, canine heartworm), manufacturing contaminants (e.g., protein A leaching), and blood alcohol level. Additional applications include proper dosing of blood thinners such as coumadin, and testing for markers indicative of inflammation (e.g. C-reactive protein). The diagnostic instruments disclosed herein may perform assays for small molecules, ions, peptides, proteins, receptors, enzymes, antibodies, nucleic acids, DNA, RNA, bacteria, viruses, cells, pathogens, and soil, air, and water contaminants, or any combination thereof. These diagnostic applications are made possible by the sensitivity of the LSPR sensor chips disclosed herein. Different LSPR sensor chips and devices may be designed for different applications.

Kits:

Also disclosed herein are kits that comprise the LSPR sensor chips and devices disclosed. In some embodiments, the kits may comprise LSPR sensor chips, test strips, or devices pre-functionalized with capture antibodies and configured to perform specific diagnostic tests. In some embodiments, the kits may comprise pre-functionalized LSPR sensor chips, test strips, or devices and one or more additional assay reagents for performing specific diagnostic tests. In some embodiments, e.g. for biomedical research applications, the kits may comprise non-functionalized LSPR sensors, test strips, or devices along with coupling reagents for functionalizing the LSPR sensor surfaces with a capture antibody or other binding component of the user's choice. In some embodiments, one or more LSPR sensors may be packaged in one or more test strips or in microfluidic devices as described above. In any of these embodiments, the kits may further comprise other assay reagents, e.g. buffers, salt solutions, enzymes, enzyme co-factors, enzyme inhibitors, enzyme substrates, antibodies or antibody fragments, proteins, peptides, oligonucleotides, and the like.

Sensor Device Concepts:

FIGS. 8-10 illustrate one non-limiting example of an LSPR sensor device in which the sensor chip is integrated with fluidic features to create an assay device suitable for near-patient or point-of-care testing. FIG. 8 illustrates a manufacturing approach in which a wafer comprising, for example, six LSPR sensor chips having integrated fluidic features which may be separated from each other using, for example, conventional dicing techniques. A wafer-based manufacturing approach allows for production scale-up and the corresponding cost savings achievable through device miniaturization and high volume manufacturing.

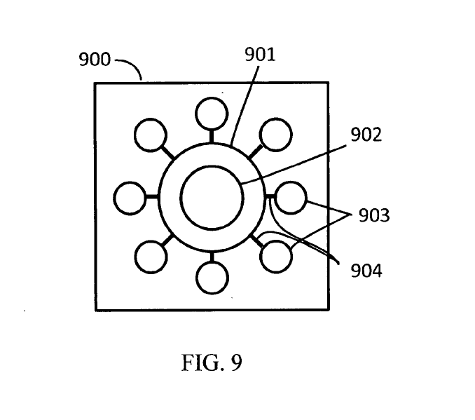

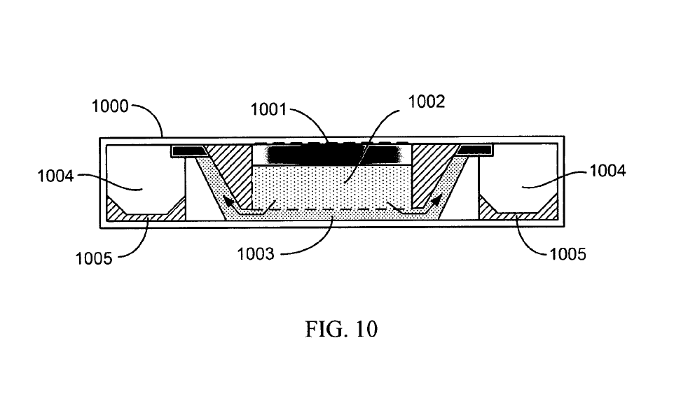

FIG. 9 provides a top view of one embodiment of an individual LSPR sensor device (900) in which the LSPR sensor chip is integrated with a microfabricated fluidics layer comprising a centrally located sample and/or reagent reservoir (901) connected to a plurality of reaction wells or chambers (903) arranged in a hub-and-spoke configuration by means of fluid channels (904), where each reaction well or chamber contains one or more LSPR sensor surfaces. IN some embodiments, the LSPR sensor chip may further comprise microfabricated pumps and valves. In some embodiments, a mechanical piston (902) may be used to drive fluid flow from the sample and/or reagent well into peripheral reaction wells or chambers. In some embodiments, a mechanical actuator (902) may exert force on a flexible membrane that seals the sample and/or reagent chamber (901). In some embodiments, positive pressure may be exerted on sample wells and/or reagent reservoirs, e.g. using a pneumatic device, to control fluid flow through the device. In some embodiment, application of vacuum to sample wells and/or reagent reservoirs may be used to control fluid flow through the device. In some embodiments, the sample and/or reagents may be placed in the central reservoir and allowed to wick through the connecting fluid channels to the reaction wells or chambers by means of capillary action. In some embodiments, the sample may be pipetted onto a filter membrane that seals the sample and/or reagent chamber, thereby providing for separation of the analyte(s) of interest from particulate contaminants. LSPR sensor chips and sensor devices may have a length of, for example, about 10 mm, about 15 mm, about 25 mm, about 30 mm, about 35 mm, or about 40 mm; a width of about 10 mm, about 15 mm, about 25 mm, about 30 mm, about 35 mm, or about 40 mm; and a thickness of less than 1 mm, about 1 mm, about 2 mm, about 3 mm, about 4 mm, about 5 mm, or more than 5 mm. For example, an LSPR sensor device may have dimensions of about 25 mm in width, 30 mm in length, and 4 mm in depth. LSPR sensor chips and sensor devices may come in a variety of different shapes and sizes. For example the LSPR sensor shape may be circular, elliptical, hexagonal, etc.

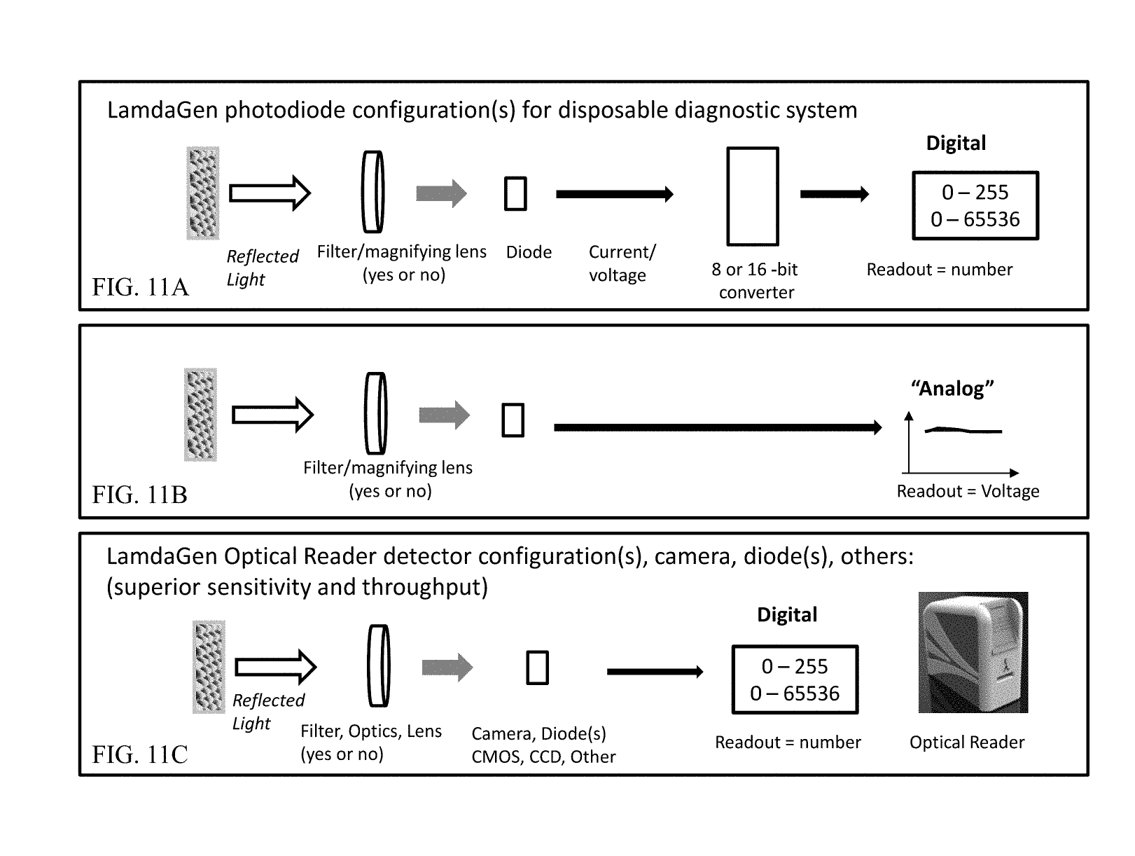

FIG. 10 provides a side cross-sectional view of one embodiment of an individual LSPR sensor device (1000) in which the LSPR sensor chip is integrated with a fluidics layer comprising a centrally located sample and/or reagent reservoir (1002) connected to a plurality of reaction wells or chambers (1004) arranged in a hub-and-spoke configuration by means of fluid channels (1003), where each reaction well or chamber contains one or more LSPR sensor surfaces (1005). Other sensor chip designs may contain another reservoir and additional reaction wells, such that each LSPR sensor chip may contain multiple reservoirs and multiple reaction wells. In some embodiments, a mechanical piston (1001) may be used to drive fluid flow from the sample and/or reagent well into peripheral reaction wells or chambers. In some embodiments, integrated components, for example, microfabricated valves, may be included for switching the fluidic conduits on and off. In some embodiments, one or more of the reaction wells may be connected to multiple reservoirs through multiple conduits. In some embodiments, the reaction chambers are staggered in different layers such that there is a clear path from each of the reaction chambers to the detector. Thus, the light reflected from an LSPR sensor surface in each reaction chamber will not be blocked by another reaction well before reaching the detector.

In some embodiments, the reaction wells are visible and open on the top surface of the LSPR sensor chip. In some embodiments, the top of the reaction wells may be sealed with a scatter-free polymer sheet, glass, or other optically transparent material, to form sealed reaction chambers while still allowing reflected light to be transmitted and detected. The bottom of the reaction wells may also comprise optically transparent material, if it is desired to detect and measure light transmitted through the LSPR sensor surface. If it is desired to detect and measure reflection, the bottom of the reaction well may be reflective. Thus, light may pass through the top of the reaction well, reflect from the bottom of the reaction well, and pass through the top of the reaction well. A detector can be placed at the same side of the reaction well as the light source for detecting reflection, or the detector can be placed at the opposite of the reaction well as the light source for detecting transmission.

The LSPR sensor chip along with its components may be fabricated from glass or silicon according to, for example, methods used to fabricate semiconductors. Alternatively, the LSPR sensor chips may be fabricated from polymer materials using techniques such as injection molding.

FIGS. 11A-C illustrates different optical detection configurations for use in portable, optionally disposable, LSPR devices and systems for near-patient and point-of-care testing environments. FIG. 11A illustrates an optical design using a minimal number of components in which light reflected from an LSPR sensor surface is optionally filtered, imaged, and/or collimated using bandpass filters and lenses and detected using a photodiode. The current generated by the photodiode in response to light is converted to voltage and digitized using, for example, an 8-bit or 16-bit converter to provide a digital output signal. FIG. 11B illustrates a similar optical design in which the current generated by the photodiode in response to light is converted to voltage and read in analogue mode. Such designs may be suitable for portable, hand-held, and wearable (potentially disposable) LSPR sensor devices. FIG. 11C illustrates an optical design in which more sophisticated detectors, e.g. CCD cameras, CMOS sensors or cameras, photodiodes, or photodiode arrays, are used to detect light reflected from an LSPR sensor surface, and read digitally. Such designs may be suitable for use in portable, hand-held or bench-top instruments or readers that interface with LSPR sensor chips and devices.

Sensor Chip, Device, and Reader Concept:

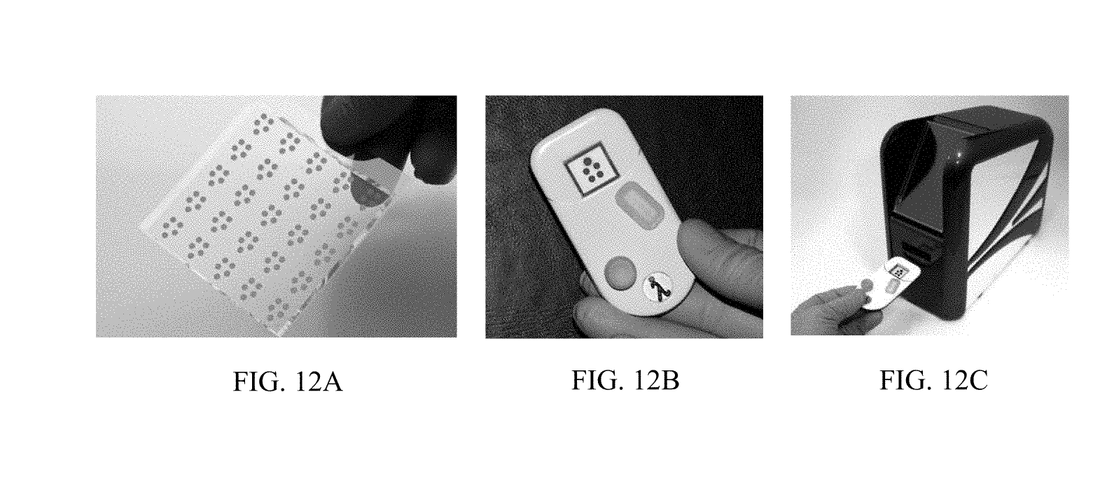

FIGS. 12A-C illustrate a system concept in which LSPR sensor chips are manufactured in wafer format (FIG. 12A), diced into individual sensor chips, and packaged in a test cartridge (FIG. 12B) that interfaces with an optical reader (FIG. 12C). FIG. 12A shows a wafer comprising a plurality of LSPR sensor chips, wherein each LSPR sensor chip comprises 5 individual sensor surfaces thereby enabling multiplexed testing. In some embodiments, some of the individual sensor surfaces on the LSPR sensor chip may be used as reference sensors or for performing assay controls. Often, the LSPR sensor chips will be packaged in a test cartridge (FIG. 12B), wherein the test cartridge may comprise fluid channels and other fluidics components for delivery of samples or assay reagents to the LSPR sensor surfaces, as well as assay reagent reservoirs containing pre-packaged assay reagents. Pre-packaged assay reagents may be stored within the test cartridges in any of a variety of formats, including but not limited to, solution phase, lyophilized (freeze-dried), or in a stabilized formulation to preserve shelf-life. The assay test cartridge may be a passive device, in which sample and/or assay reagents wick through fluid channels within the test cartridge by means of capillary action, or it may be an active device, in which fluid actuation and mixing steps are performed by pumps, valves, and other active components incorporated into the test cartridge, or are controlled through the interface with the reader instrument. Following addition of a sample to the sample well of the test cartridge, the test cartridge is inserted into the optical reader (FIG. 12C), where the assay reaction is allowed to proceed on the sensor surface and the result is optically read. In some embodiments, the LSPR sensor chip or assay test cartridge may comprise a sample collection device, e.g. a capillary, or micro- or nanoscale-needles, for drawing in the sample to be tested. In some embodiments, the assay reaction is performed within the test cartridge prior to inserting the test cartridge into the optical reader. In some embodiments, the assay reaction is performed after inserting the test cartridge into the optical reader. In some embodiments, multiple data points are measured by the optical reader to provide kinetic data that tracks the progress of the assay reaction over time.

Sensor Card, Optical Device, and Mobile Phone System Concept:

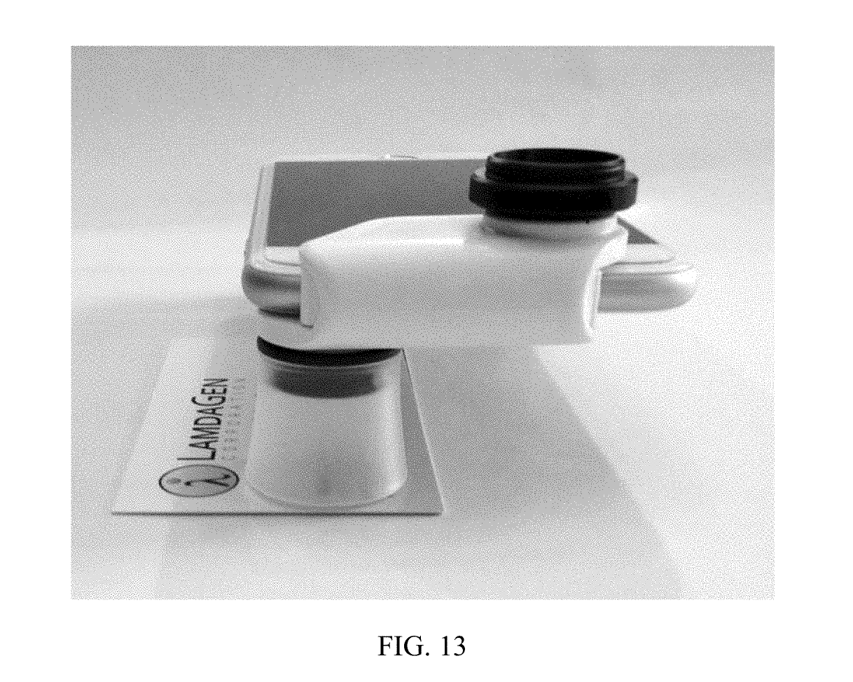

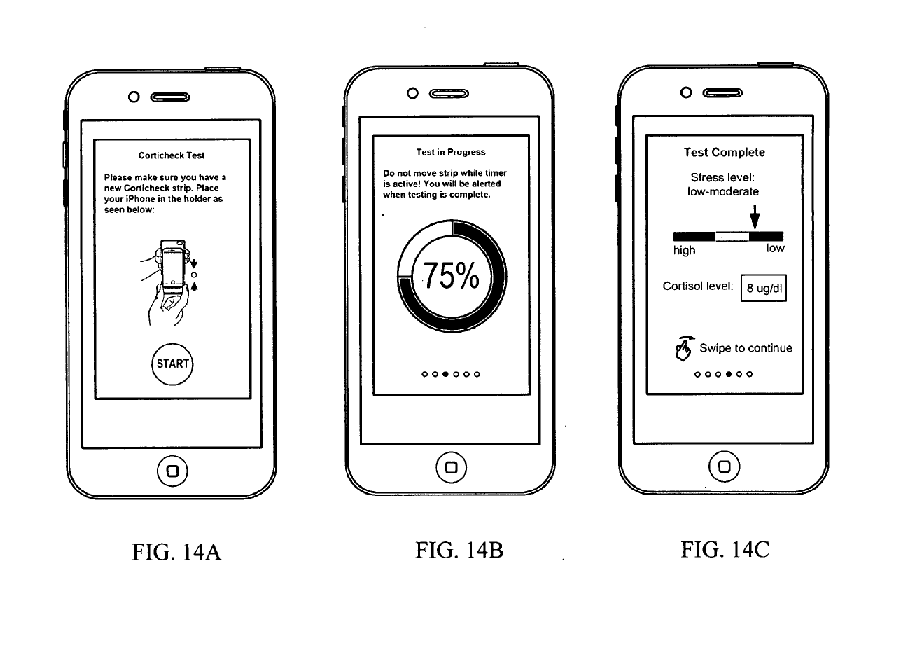

FIGS. 13 and 14 illustrate a hand-held, LSPR-based point-of-care diagnostic test system concept in which LSPR sensor chips are integrated into a credit card-like format for use in simple, one-step assays, and the sensor card is read using a simple optical attachment that interfaces with a mobile phone or other smart device (e.g. a smart phone, a tablet computer, or any other smart device) comprising a camera (FIG. 13). The optical attachment would include a compact light source, imaging optics, optional band-pass filters, and, for example, a CMOS image sensor. In some embodiments, the mobile phone's built-in camera may serve as the detector. In this concept, the mobile phone or smart device may also act as the processor which acquires and processes the data from an LSPR sensor chip designed to perform a specific diagnostic test, e.g. a cortisol test (FIGS. 14A-B), and displays the test result (FIG. 14C). In some embodiments, the mobile phone application is further configured to upload the test results to an internet cloud-based database and/or send a message to a designated family member or healthcare provider. In some embodiments, the mobile phone is configured to upload the test results to an internet cloud-based healthcare software application. Potential advantages of such a test system include more frequent testing when needed, faster times to results, improved patient compliance with testing and therapeutic routines, and improved healthcare outcomes. One non-limiting example of a rapid assay that may be implemented using LSPR sensors and a mobile phone-based system is a cortisol assay. LSPR sensors would be incorporated into a microfluidics format within a credit card-sized “sensor card”. Application of a drop of blood to the sample well on a disposable card would initiate the assay in which, for example, capillary action drives fluid flow through a filter membrane, thereby separating blood cells from plasma, which would subsequently undergo diffusional mixing with detection reagents stored within the device and be delivered to the LSPR sensor surface. In some embodiments, the disposable sensor card may comprise a sample collection device, e.g. a small capillary tube for drawing a sample to be tested into the device. In some embodiments, the disposable sensor card may further comprise a lancet for piercing skin. Changes in the reflective properties of the sensor surface resulting from presence of the analyte in the sample would be read by an optical attachment that interfaces with a mobile phone or smart device, as described above. Examples of data for a cortisol assay using a competitive immunoassay format and LSPR sensors for detection are presented below.

Wearable LSPR Sensor Device Concepts:

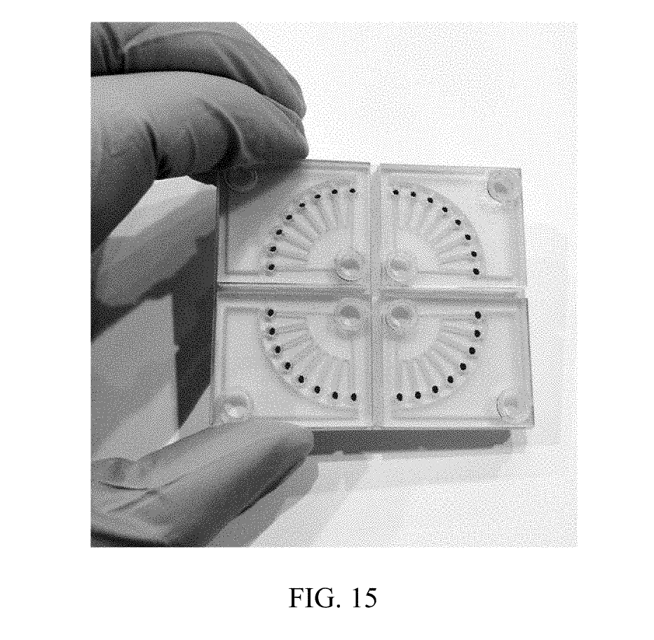

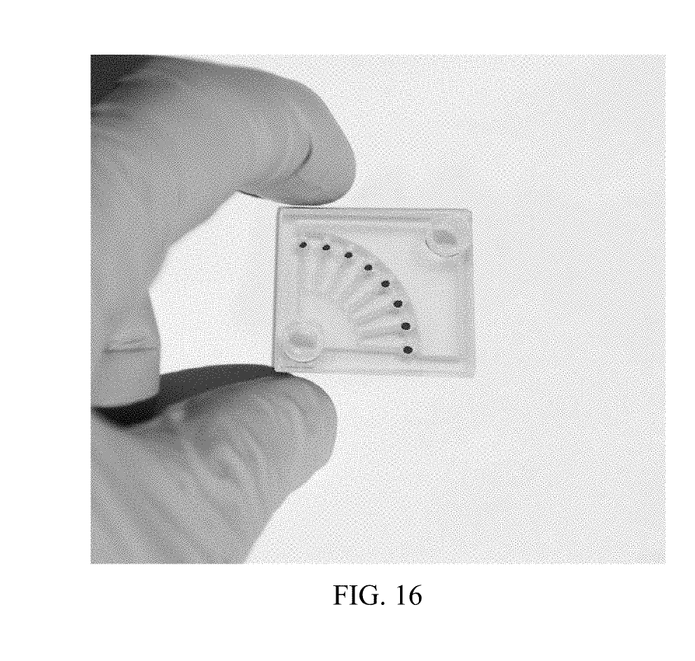

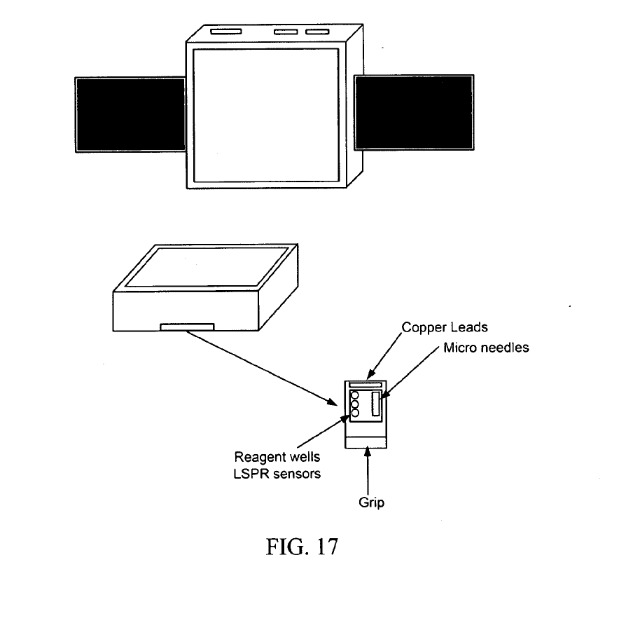

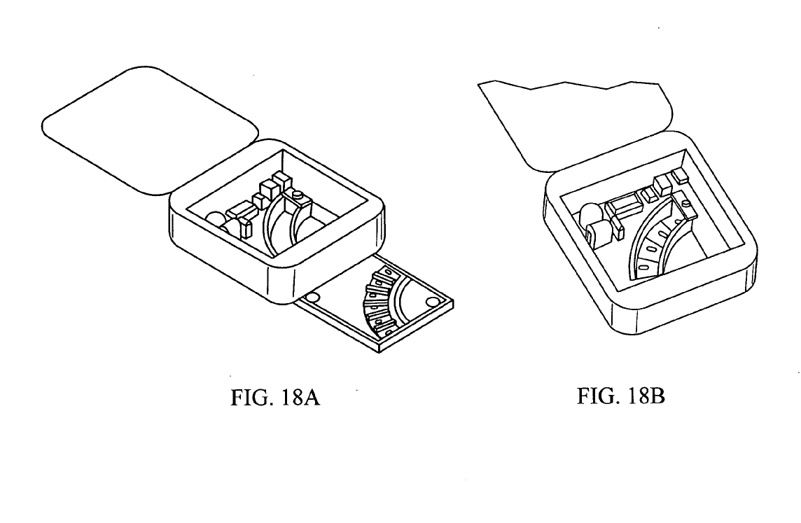

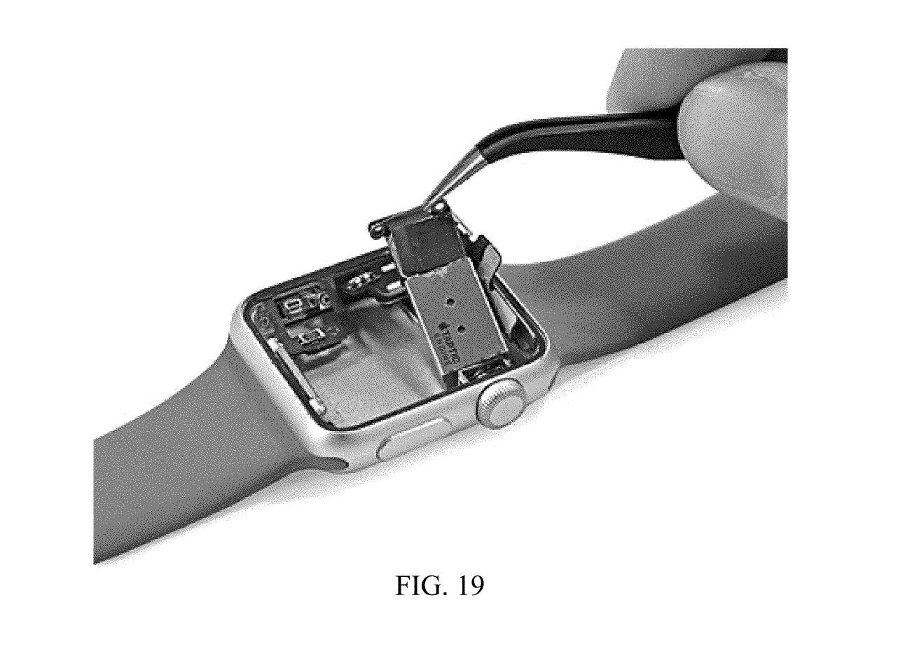

FIGS. 15-19 illustrate one non-limiting example of a wearable device concept for using LSPR sensors to perform point-of-care testing in a periodic or continuous testing mode. FIG. 17 illustrates one embodiment of an LSPR sensor device that is configured as a wrist device. The wrist device may be attached to wrist bands, creating a wearable wrist device. The LSPR sensor chip and wrist device may interface with each other through a slot in the wrist device adapted to receive the LSPR sensor chip. In some embodiments, the LSPR sensor chip is a disposable component intended for one-time use while the wrist-device may be suited for repeat use. In the example illustrated in FIG. 17, the LSPR sensor chip is rectangular in shape and may be sized so that a user may handle the LSPR sensor chip comfortably. The sensor chip may comprise one or more sample and reagent wells, reagent reservoirs (not shown in FIG. 17), fluid conduits (not shown in FIG. 17), and one or more reaction chambers that incorporate LSPR sensors, as well as micro-needles, a grip, and copper leads for making electrical contacts between the sensor device and the wrist device. The LSPR sensor chip may further include alignment features for aligning and securing the LSPR sensor device precisely within the wrist device. The alignment feature may be an off-center circular depression or raised feature. FIGS. 15 and 16 show photographs of prototype LSPR sensor devices (in wafer format, and diced into individual sensor devices, respectively), wherein the sensor devices comprise a plurality of fluid channels and reaction chambers, each comprising an LSPR sensor, as well as sample and reagent reservoirs. FIGS. 18A and B provide additional views of the wearable wrist device (band not shown). LSPR sensor chips comprising multiple reaction chambers, each containing one or more LSPR sensors, slides into the wearable housing and interface with miniaturized optical and electronic components. Pressing on the top of the wrist device activates one or more micro- or nano-scale needles which penetrate the skin of the user and draw a nanoliter to microliter scale sample of blood or interstitial fluid. Blood or interstitial fluid drawn through the micro- or nano-needles is optionally filtered to remove blood cells or other particulates (e.g. using microfabricated filtration features), optionally mixed with assay buffers or detection reagents (e.g. added manually by the user, or using reagents pre-loaded in the device), and delivered to the one or more LSPR sensor surfaces. Microfabricated optical components, e.g. light emitting diodes (LEDs) and photodiodes, incorporated into the wrist device provide light sources and detectors for interrogating the LSPR sensor surfaces, while microprocessors incorporated in the wrist device acquire and process the assay data, display the test results, and optionally transmit the test results to an external computer or internet-based database. FIG. 19 further illustrates the wearable wrist device concept.

Referring back to FIG. 17, the sample to be assayed may be introduced to the LSPR sensor chip through the use of micro-needles. For example, when the LSPR sensor chip is interfaced with the wearable wrist device and worn by the user, the LSPR sensor chip may be flush with the bottom of the wearable wrist device such that the micro-needles contact the user's skin. To activate the micro-needles, the user may depress a button on the wearable wrist device for a period of time that ensures the depression was not accidental. For example, the user may be required to depress the button for a period of 5 seconds, 10 seconds, 15 seconds, or more, in order to active the micro-needles. The micro-needles may prick the user's skin, drawing blood. The blood may then be transported to the reaction wells, where the assay takes place.

In some embodiments, the LSPR sensor chip and housing device may be set up for substantial real-time monitoring. Thus, the micro-needles may prick the user's skin to draw blood every minute, every 10 minutes, every 30 minutes, every hour, every two hours, or any other applicable frequency. Every time blood is drawn, the blood sample may be introduced to the same LSPR sensor chip because one LSPR sensor chip may contain a plurality of reaction wells or chambers where the assay takes place.

In other embodiments, the sample may be introduced to the reaction wells through an external sample collection mechanism. For example, the user may utilize an external device to collect bodily fluid and deposit a drop or less of the bodily fluid onto the LSPR sensor chip. The sample may be any bodily fluid, such as blood, sweat, tears, urine, and saliva, or other fluid (e.g., contaminated water).

In some embodiments, the sensor chip design may include one or more reactions wells and reservoirs organized in distinct layers. One or more reaction wells may be used to run assays initially, and a second set of reaction wells may be used for confirmation to ensure against false positives and false negatives. The sample assayed in the second set of reaction wells may be different from the sample assayed in the first set. The sample assayed in the second set of one or more reaction wells may be assayed for confirmation purposes only.

In some embodiments, the LSPR sensor chips run single tests. In other embodiments, the LSPR sensor chips are multi-paneled or multiplexed, such that a different type of assay may be run in each reaction well. Thus, different reaction wells may contain different antibodies, DNA for running DNA assays, RNA, bacteria, viruses, cells, ligands, proteins, oligos and aptamers, fragment of organic matter, and so forth that are immobilized in the reaction wells. Such multi-paneled reaction assays may be useful because diagnosis of some diseases may require detection of more than one biomarker. Thus, at least two biomarkers may be necessary to identify a disease. Multi-paneled LSPR sensor chips allow assays for multiple biomarkers to be run on the same chip. As another example, a multi-paneled reaction assay may be useful for determining which type of flu a user has. A user may experience flu-like symptoms and desire to find out what type of flu he/she has. To do so, the user may deposit a sample on a multi-paneled LSPR sensor chip which can assay multiple types of flus. Thus, a user may be able to find out what type of flu he/she has using only one LSPR sensor chip. As another example, one LSPR sensor chip may be multi-paneled to assay for multiple drugs of abuse.

In some embodiments, LSPR sensor chips may comprise one or more reservoirs that may contain pre-loaded, reagents, diluents, secondary antibodies that are un-conjugated, secondary antibodies conjugated with enzymes, secondary antibodies conjugated with mass-enhancing beads, secondary antibodies conjugated with plasmonic moieties, and the like. In some embodiment, LSPR sensor chips may comprise fluid channels containing lyophilized assay reagents such that the reagents are solubilized when sample and/or assay buffers are added to the device. Often, the LSPR sensor chips may comprise one or more waste reservoirs for storing waste products resulting from running an assay.

In some embodiments, there is a membrane that serves as a filter placed on top of the reaction well. In some embodiments, the sample to be assayed may be deposited onto the LSPR sensor chip by depositing the sample directly over the reaction well on top of the filter. The filtration membrane may be designed to filter out unwanted particles according to size. For example, the filtration membrane may contain appropriately sized holes that only allow smaller sized particles to filter through to the reaction wells. Unwanted particles may include cells, salt crystals, insoluble precipitates, or other particulates which may interfere with the assay or clog the fluid conduits. A sample may contain one or more molecules of interest which may be separated by the membrane. Thus, different types of molecules may filter through to different reaction wells and membranes of different porosity may enable the concurrent analysis of more than one analyte in a sample

In some embodiments, when the sample to be assayed is blood, the red blood cells and white blood cells may be filtered out, such that only the blood plasma filters through. The blood cells may be undesirable because they may clog the conduits or otherwise interfere with the assay. However, in some embodiments, filtering may not be necessary and blood cells may still be introduced into the system if, for example, diluents and/or anti-coagulation agents are added to the blood. Thus, some embodiments do not include a filter on top of the reaction wells.

In some embodiments, the sample is introduced by depositing it over a reservoir instead of or in addition to a reaction well. The LSPR sensor chip may contain one or more reservoirs especially adapted to receive samples. The sample reservoirs may or may not include membranes placed on top of the reservoirs depending on whether filtering is desired.

The sample may also be introduced to the LSPR sensor chip by depositing it to a reservoir containing diluent. The diluent reservoir may or may not contain a membrane depending on whether filtering is desired. The sample may be mixed with the diluent in the reservoir, and then the diluted sample may be introduced into the reaction wells. The sample may also be deposited to a reservoir containing both diluent and secondary antibodies (which may be un-conjugated, conjugated with enzymes, conjugated with mass-enhancing beads, or conjugated with plasmonic moieties). The sample may be mixed with the diluent as well as the secondary antibodies, and then the mixed sample may be introduced into the reaction wells.

Filtration may be achieved by mechanically pressing down on the sample with, for example, a piston. When the piston exerts pressure on the sample, the smaller particles may be forced through the filtration membrane while the larger particles do not pass through the filtration membrane. Filtration may also be achieved without mechanically pressing down on the sample. For example, filtration may be achieved by gravitational forces or through negative pressure applied from the side of the filtration membrane opposite where the sample lies.

In some embodiments, the sample reservoir is sealed. For example, it may be sealed with a self-sealing septum. In this embodiment, samples may be introduced into the reservoir by puncturing the self-sealing septum with a needle and injecting the sample into the sample reservoir. In other embodiments, the sample reservoir may be sealed with a membrane, cap, lid, or the like. To introduce the sample into the reservoir, the cap or lid can be removed.

Referring to FIG. 10, after the reservoir receives the sample (which may be diluted and/or filtered and/or mixed with secondary antibodies), the sample may be transported to the reaction wells by activating a piston contained in the housing device. A piston mechanism may be coupled to the reservoir to actuate flow of the sample through the one or more fluid conduits. The piston may mechanically, push down on the fluidic sample in the reservoir, pushing the sample out the bottom and through the conduits. Referring to FIG. 10, the fluidic sample may then be siphoned up the conduits through capillary action and into the reaction wells. In some embodiments, the conduits are angled upward at around 10 degrees relative to the base of the reservoir, as illustrated in FIG. 10.

The fluidic sample may be transported through the conduits and into the reaction wells through other means as well besides those utilizing capillary action. For example, pumps and valves may be utilized to ensure one way flow of fluids from the reservoir to the reaction wells. Referring to FIG. 10, valves may be included at the juncture where the fluid conduits and the reservoirs meet. The valves may be membranes which contain the fluid in the reservoir and prevent the fluid in the reservoir from flowing into the reaction wells until the desire time. At the desired time, the membrane valve may be ruptured by applying pressure to it (e.g., by utilizing a piston that presses down into the reservoir and increases the pressure of the fluid which, in turn, exerts pressure on the membrane valve). Thus, when the membrane value is ruptured, fluid may flow from the reservoir through the fluid conduits and into the reaction wells. Another type of valve that may be used is a silicon membrane valve. The silicon membrane valve may be a one-way valve that opens when pressure is exerted on it from one side but not the other. For example, the silicon membrane valve may open when pressure is exerted on the side of the valve that faces the reservoir, but the silicon membrane may not open when pressure is exerted on the side of the valve that faces the conduit. When pressure is returned to normal, the silicon membrane valve may return to its closed state. In some embodiments, two or more silicon membrane valves incorporated into the sensor chip may have different requirements for the amount of opening force required, and therefore application of increasing force by the piston may open the two or more valves in a pre-defined, sequential order. Other examples of valves that may be utilized include solenoid valves, pinch valves, and pneumatic valves.

The sample may be introduced into different reaction wells or chambers at different times by controlling the length, hydrophobicity, and/or capillary properties of the channels.

The size and shape of the conduits, as well as the speed and pressure with which the piston pushes down on the fluidic sample, may be designed such that flow into the reaction wells is laminar. In some embodiments, the length of the conduits may be around 5 mm and the diameter of the conduits may be around 0.5 mm. The amount of sample delivered to the reaction wells may be controlled by controlling how deeply the piston is pushed into the reservoir. In this manner, the sample may be introduced into the reaction wells.

As mentioned previously, the LSPR sensor chip may also contain reservoirs for storing other fluids or reagents using in performing the assay. The different fluids in the different reservoirs may be introduced into the reaction wells according to the type of assay to be run. For example, for assays utilizing the ELISA assay format, after the sample is introduced into the reaction wells, a diluent from a diluent reservoir may be introduced in order to wash the reaction wells. Afterward, secondary antibodies conjugated with enzymes can be introduced into the reaction wells from the enzyme-conjugated secondary antibody reservoir. Next, diluent can again be introduced in order to wash the reaction wells. Next, the reagents, such as substrates that can be enzymatically converted to an insoluble precipitate, can be introduced into the reaction wells from the reagent reservoir. Thus, LSPR sensor chips that utilize the ELISA assay format may contain a sample reservoir (or the sample may be deposited directly in the reaction well without a sample reservoir; additionally the sample reservoir may include diluent to be mixed with the sample), a diluent reservoir, a reservoir for secondary antibodies conjugated with enzymes, a reagent reservoir, and a waste reservoir. In some embodiments, a wash step may not be necessary for blood samples or other samples which are sufficiently diluted. Thus, some LSPR sensor chips may omit a diluent reservoir where the blood sample or other sample is sufficiently diluted before it is introduced onto the LSPR sensor chip.

In another embodiment, instead of introducing the different fluids into the reaction wells sequentially, the different component fluids may be pre-mixed and introduced into the reaction wells in one step. For example, the sample, diluent, and secondary antibodies (which can be un-conjugated, conjugated with an enzyme, conjugated with a mass-enhancing particle, or conjugated with a plasmonic moiety), may be pre-mixed in a reservoir. Next, the pre-mixed fluid containing the diluted sample and secondary antibodies may be introduced into the reaction wells. In these embodiments, the LSPR sensor chips may contain a reservoir containing diluent and secondary antibodies, which can be mixed with the sample when the sample is introduced into that reservoir. Further, the LSPR sensor chips may also contain additional diluent reservoirs for rinsing, as well as waste reservoirs.

For assays utilizing the plasmon-plasmon coupling sandwich immunoassay format, a rinse step may not be necessary. Thus, after the sample is introduced into the reaction wells, the secondary antibodies conjugated with plasmonic moieties may be introduced into the reaction wells from the reservoir holding the secondary antibodies conjugated with beads. In another embodiment, the sample may be deposited into a reaction well containing secondary antibodies conjugated with plasmonic moieties, and then the sample may be mixed with those secondary antibodies. The mixed fluid may then be introduced into the reaction wells. Thus, LSPR sensor chips utilizing the plasmon-plasmon coupling sandwich immunoassay format may constitute a one-pot assay. These sensor chips may contain a waste reservoir, and a reservoir for storing secondary antibodies conjugated with plasmonic moieties, wherein that same reservoir may also be configured to contain a sample (or the sample may be deposited directly into the reaction well).

As new fluids are introduced into the reaction wells, pre-existing fluids may be pushed out through a waste conduit and into a waste reservoir. In order to prevent back flow from the waste reservoirs, valves may be incorporated into the conduits connecting the waste reservoirs and the reaction wells. Optionally, the capillary flow may be designed to prevent back-flow from the waste reservoir.

In some embodiments, the reservoirs may be partitioned into chambers, with each chamber resembling the shape of a slice of pie. Each chamber may be connected to a single conduit leading to a single reservoir. Each chamber may contain a different type of fluid. When the piston depresses into the reservoir, the fluid in each chamber may be dispersed to its respective reaction well.

In some embodiments, the conduits may be lined with lyophilized secondary antibody conjugates (or other assay reagents), which are picked up by sample when the sample is pushed through the conduits and into the reaction wells. Thus, when the sample is pushed through the conduits and into the reaction wells, the sample may pick up the lyophilized secondary antibodies, and the secondary antibodies along with the sample may be introduced into the reaction wells. In this manner, the sample and the secondary antibody conjugates may be introduced into the reaction wells in an efficient manner.

In order to control the flow of different fluids into different reaction wells at different times, microfluidic open/closed valves or gates may be used.

In addition to a wrist device, LSPR sensor chips may interface with a variety of different types of housing devices. Other examples of portable wearable devices include a necklace, a belt, a patch, or a leg strap. Wearable devices may include any device that can be attached to a human or animal. In other embodiments, the housing device may not be specifically adapted to be wearable but may be portable, hand-held, and/or mobile so that the housing device can be conveniently carried around by the user. In other embodiments, the housing device may be a bench top device that may be suitable for placement in doctor's offices or other clinical locations.

In some embodiments, LSPR sensor chips and/or housing devices can be integrated into consumer devices that contain various other functions. For example, in some embodiments, the LSPR sensor chips and/or housing devices can be attached to or integrated into a cell phone, a tablet, a laptop computer, a desktop computer, earphones, or exercise equipment. Additional examples of systems which may incorporate LSPR sensor chips and sensor devices include automobiles, trucks, or other types of transportation vehicles and systems, as well as in robots, drones, and the like. Any and all of these devices can include a slot specially adapted to interface with the LSPR sensor chip. In some embodiments, the sensor housing device communicates wirelessly with the consumer device. The sensor housing device may also be connected to the consumer device through external wires/cables such as USB cables. In some embodiments, the sensor housing device is an integral part of the consumer device.

The housing device may be integrated with any consumer product, including those that do not ordinarily have electronic components. For example, the housing device can be integrated into a helmet, a piece of furniture, or bullet-proof vests.

In some embodiments, the housing device can be integrated into the steering wheel of a car. For example, a LSPR sensor chip may be interfaced with the housing device installed on the steering wheel. When the user places his/her hand on the wheel, bodily fluid such as sweat (e.g. perspiration from the fingertips) may be collected from the user and deposited onto the LSPR sensor chip. The housing device and the LSPR sensor chip may then run an assay and the results of the assay may be displayed on the car's dashboard. This application may be useful for detecting blood alcohol levels, glucose levels, and drugs of abuse.

In some embodiments, the housing device is a stand-alone housing device that does not require components from other devices (other than the LSPR sensor chip) to run and process the assay. In other embodiments, when the housing device is integrated into consumer devices, components of the consumer devices may be utilized to run and process assays. For example, when a housing device is integrated with a mobile device, the microprocessor, the power source, the display, and/or the wireless communication mechanism (e.g., Bluetooth, WiFi) of the mobile device may be utilized to process and display the assay results.

The components of a housing device may include one or more white light sources (e.g., an LED white light source), one or more band-pass filters or monochromators, one or more detectors, a microprocessor, a power source, a display, an on-off switch, and a wireless communication mechanism such as Bluetooth or WiFi. Example detectors include a miniaturized spectrometer, a photodiode, a pin diode, an avalanche diode, a CCD sensor, a CMOS sensor, or any other optical detector. These components allow for instrumental simplicity.

In some embodiments, the detection method may utilize a camera and high resolution digital imaging, as described above in the section ELISA-based LSPR sensors coupled with digital imaging” to improve assay sensitivity. In some embodiments, the camera may be a cell phone camera. The lens of the camera may be as small as 1 mm in diameter.

The beam spot size of the white light source may be controlled by changing the numerical aperture of the optical assembly used to deliver the light to the sensor surface. Some embodiments may include a separate white light source for each reaction well. The beam spot size of each white light source may be tailored to match the size of the reaction wells, to avoid wasting light and maximize intensity, while still covering areas of interest.

In some embodiments, the white light may pass through a band-pass filter so that only a smaller portion of the light spectrum reaches the detector. In these embodiments, a pin diode may be used as a detector to monitor changes in light intensity. By utilizing a band-pass filter, simplicity and cost reduction of the optical reader design can be achieved.

In some embodiments, the detector may be used to monitor reflected or transmitted light at two or more wavelengths, which is accomplished by passing the white light source through two or more band-pass filters. The wavelengths of light used to monitor analyte-induced changes in reflected or transmitted light can be determined by running laboratory tests using a full spectrometer to identify the plasmon absorption peak wavelength, and determining which wavelengths exhibit the greatest change in intensity (or ratio of intensities), and therefore are most important to monitor.

In some embodiments, the white light source and/or the detector may be coupled to a scanner that moves the white light source and/or detector across the reaction wells at various speeds. By including a scanner, it may not be necessary to include multiple light sources. The white light source and/or detector may be passed across all the reaction wells once, twice, or any number of suitable times, in order to determine the amount of analyte at given points in time.

Operation of the LSPR Sensor Chip and Housing Device:

In operation, a sample may be collected and deposited onto the LSPR sensor chip. In some embodiments, the sample is collected using micro-needles. In other embodiments, the sample may be collected using an external device. Next, the sample may be introduced to the reaction well by depositing the sample directly into the reaction well, or by depositing the sample into a reservoir. The reaction well and/or the reservoir may contain a membrane over the top of the reservoir that serves to filter out unwanted components of the sample, such as red and white blood cells from a blood sample. In some embodiments, the blood is not filtered because the blood may be sufficiently diluted before entering the reaction well and/or because anti-coagulants may be present in the blood. If the sample is deposited into the reservoir, a piston may depress into the reservoir in order to push the sample out the bottom and through the conduits into the reaction wells.

In some embodiments, the sample is deposited into a reservoir that contains a diluent and secondary antibodies (which may be un-conjugated, conjugated with enzymes, conjugated with mass-enhancing beads, or conjugated with plasmonic moieties). For the enzyme assay format, the secondary antibodies may be conjugated with enzymes. For the plasmon-plasmon coupling sandwich immunoassay format, the secondary antibodies may be conjugated with plasmonic moieties. The reservoir may also have a filtration membrane on top of the reservoir (i.e. at the inlet of the reservoir) or otherwise incorporated into the reservoir design. Thus, the sample may be filtered, and the filtered sample may then enter the reservoir containing diluent and secondary antibodies. The filtered sample may then mix with the diluent and the secondary antibodies. Next, the piston may depress into the reservoir, pushing the filtered diluted sample mixed with the secondary antibodies out the bottom, through the conduits, and into the reaction wells. During this process, pressure may be exerted on valves located at the juncture where the reservoir and conduit meet, thus opening the valves and allowing the fluid to flow through. In some embodiments, the one or more valves incorporated into the sensor chip may have different requirements for the amount of opening force required, and therefore application of increasing force by the piston may open the one or more valves in a pre-defined, sequential order. The secondary antibodies may then be allowed to incubate in the reaction wells for some time, in order to allow the ELISA reaction, or plasmon-plasmon coupling reaction, to take place. In some embodiments, the reaction well may be rinsed with diluent from a diluent reservoir.

White light may be directed at the reaction wells before, during, and after the plasmon-plasmon coupling or the ELISA reaction takes place. A detector may detect a shift in the optical absorption peak before, during, and after the plasmon-plasmon coupling or the ELISA reaction takes place. The shift may be used to determine the amount of analyte present in the sample. The results may then be output to the user through a display on the housing device or through a display on a device with which the housing device is integrated or attached to, such as a mobile phone. In some embodiments, the display may show the amount of analyte present in the sample. For example, the display may show the blood sugar level of a user. In other embodiments, the display may show whether the analyte is present or not. For example, the display may show whether or not a user has the flu. In some embodiments, analyte may be measured substantially in real-time. Thus, the user may receive as output information the amount of analyte as a function of time. In some embodiments, the results of the assay are transmitted wirelessly to a doctor, pharmacist, or other health care professional. The health care professional may then prescribe a recommended course of action to the user.

Example—Competitive Assays and Cortisol Detection:

A competitive assay is a well-known immunoassay technique that is particularly well suited for detection of small molecules with molecular weight <1000 Daltons, and is therefore a useful assay technique for diagnostic tests. In one embodiment, an antibody specific to a target antigen is spiked into a sample. An antigen similar to the one to be detected in the sample is immobilized on the biosensor such that the immobilized antigen and the target antigen in the sample compete for antibody binding. If antigen present in the sample binds to the antibody in solution, it prevents the antibody from binding to the antigen immobilized on the sensor surface, thereby reducing the biosensor signal. A competitive assay is therefore an inverse assay relative to a traditional immunoassay, as the competitive assay exhibits a large signal at low antigen concentrations and a small signal at high antigen concentrations.

The LSPR biosensors disclosed herein are well suited for performing competitive assays, and could be incorporated into a variety of portable bench-top, hand-held, mobile phone-based, or wearable diagnostic test systems. This example describes a highly sensitive and rapid competitive assay for the detection of cortisol in saliva or serum.

Cortisol is a stress related biomarker which is the end product of the hypothalamic-pituitary-adrenal axis. Cortisol levels vary from one individual to another, and are also time-dependent as they follow a natural circadian cycle with low levels at night (˜100 pg/mL) and higher levels in the morning (˜5 ng/mL). In addition to the natural cycle, cortisol concentrations peak at levels higher than their typical values about 15 min after the onset of a stress-inducing stimulus.

Various competitive assay platforms have been developed to measure the cortisol evolution over the course of the day for an individual. The most sensitive tests (LOD ˜37 pg/mL) are based on an ELISA assay format but require more than 2 hours to complete, and are therefore performed in central labs. On the other end, rapid assays are available that provide a quantitative result after ˜20-25 minutes using a lateral flow assay format and a chromophore particle (e.g., phosphorescent microparticle or colloidal gold) as the signal generator. However, the available rapid tests lack the sensitivity of the competitive ELISA assays, with LODs in the ˜1 ng/mL range.

There is currently an unmet need for technology that allows the detection of cortisol in 20 minutes or less, with a detection limit and dynamic range that span an analyte concentration range from ˜100 pg/mL upwards. The LSPR sensor technology of the present disclosure is able to fulfill this need.

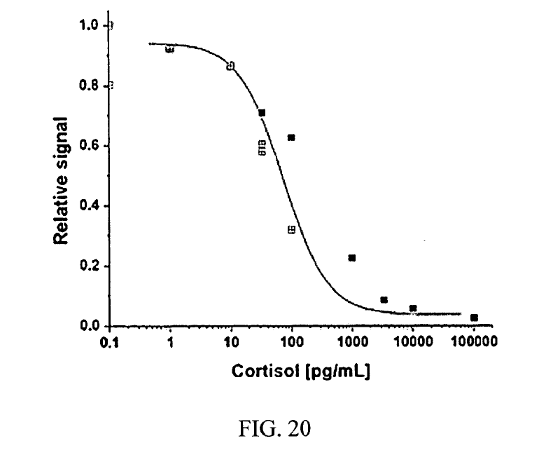

FIG. 20 shows the dose response curve for a 20 minute competitive immunoassay for cortisol that has a quantitative range of 10-3,000 pg/mL. For this assay, the LSPR biosensor surface was functionalized with BSA-cortisol. A cortisol calibration curve was determined using serial dilutions of a stock solution of 10 ug/mL cortisol in an assay buffer. The final standard concentrations of cortisol spanned the range from 1 pg/mL to 100 ng/mL.

A solution containing a 1:1 volume ratio of sample (i.e. cortisol standard for the calibration curve, and saliva or serum/plasma for the actual sample to be analyzed) and colloidal gold (OD=2) was added to the LSPR biosensor surface without pre-incubation. The colloidal gold was coated with both an anti-cortisol antibody and the enzyme alkaline phosphatase (AP). After a brief incubation, the sample was rinsed away with assay buffer.

BCIP/NBT, a substrate for alkaline phosphatase, was then added. The presence of the AP enzyme at the surface of the biosensor triggers a chemical reaction that converts the soluble BCIP/NBT into an insoluble formazan compound that deposits on the sensing surface. This generates a color change of the surface. The assay results were quantified using a prototype of a compact, portable optical reader such as those described previously in this disclosure. FIG. 20 shows examples of data for the cortisol competitive immunoassay performed using two different sensors (indicated by the grey squares and black squares respectively). The assay required 20 minutes to perform and exhibited a linear cortisol quantification range spanning from ˜10 to ˜1000 pg/mL.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A sensor chip comprising:
a) one or more reaction wells, wherein each reaction well comprises a sensor surface capable of sustaining a localized surface plasmon resonance;
b) a sample reservoir configured to contain a sample comprising an analyte; and
c) one or more fluid conduits, wherein each fluid conduit connects the sample reservoir and one of the reaction wells;
wherein the one or more sensor surfaces exhibit an analyte-induced change in optical property upon contact with the sample.

2. The sensor chip of claim 1, further comprising a primary binding component immobilized on each of the one or more sensor surface(s), wherein the primary binding component is selected from the group consisting of antibodies, antibody fragments, peptides, proteins, aptamers, molecularly imprinted polymers, biotin, streptavidin, hist-tags, chelated metal ions such as Ni-NTA, oligonucleotides, or any combination thereof.

3. The sensor chip of claim 1, further comprising at least a second sample reservoir.

4. The sensor chip of claim 1, further comprising at least one reagent reservoir.

5. The sensor chip of claim 1, further comprising at least one waste reservoir.

6. The sensor chip of claim 1, wherein the sample reservoir further comprises a filtration membrane.

7. The sensor chip of claim 1, wherein the sample reservoir is sealed.

8. The sensor chip of claim 7, wherein the sample reservoir is sealed with a cap, a flexible membrane, or a septum.

9. The sensor chip of claim 1, wherein the one or more reaction wells are sealed with an optically transparent material.

10. The sensor chip of claim 9, wherein the optically transparent material is glass or a scatter-free polymer sheet.

11. The sensor chip of claim 1, further comprising at least one microfabricated pump.

12. The sensor chip of claim 1, further comprising at least one microfabricated valve.

13. The sensor chip of claim 1, wherein a thickness of the sensor surface is about 15 nm to about 200 nm.

14. The sensor chip of claim 1, wherein the sensor surface comprises two or more layers of material.

15. The sensor chip of claim 14, wherein a thickness each layer is about 5 nm to about 100 nm.

16. The sensor chip of claim 14, wherein each layer comprises metal, noble metal, polymer, ceramic, or glass.

17. The sensor chip of claim 14, wherein a top layer has a primary binding component immobilized thereon, wherein the primary binding component is selected from the group consisting of antibodies, antibody fragments, peptides, proteins, aptamers, molecularly imprinted polymers, biotin, streptavidin, his-tags, chelated metal ions such as Ni-NTA, oligonucleotides, or any combination thereof, and wherein the top layer is a nanostructured, noble metal thin film.

18. The sensor chip of claim 1, wherein the surface comprises a nanostructured, doped or self-doped semiconductor thin film.

19. The sensor chip of claim 18, wherein the nanostructured, doped or self-doped semiconductor film is copper(I) sulphide (Cu₂₋ₓS), a doped semiconductor-based oxide (including but not limited to aluminum-doped ZnO, gallium-doped ZnO, or indium-tin oxide) or a transition metal nitride such as nitrides of titanium (TiN), of tantalum (TaN), of hafnium (HfN) or of zirconium (ZnN).

20. The sensor chip of claim 1, wherein the sensor surface comprises a nanostructured, metal thin film.

21. The sensor chip of claim 20, wherein the nanostructured, metal thin film is a nanostructured, noble metal thin film.

22. The sensor chip of claim 21, wherein the nanostructured, noble metal thin film is a nanostructured, gold thin film.

23. A device for detecting an analyte in a sample, the device comprising:
a) a substrate comprising one or more localized surface plasmon resonance (LSPR) sensors, wherein analyte molecules are immobilized on a surface of the one or more LSPR sensors; and
b) a cartridge, wherein the cartridge either partially or completely encloses the substrate, and wherein the surface(s) of the one or more LSPR sensors are accessible to addition of the sample.

24. The device of claim 23, wherein the device is configured to perform a competitive immunoassay for the detection and quantification of the analyte in the sample.

25. The device of claim 24, wherein the analyte is selected from the group consisting of a peptide, a protein, an oligonucleotide, a lipid molecule, a carbohydrate molecule, a small organic molecule, a drug molecule, or any combination thereof.

26. The device of claim 25, wherein the analyte is selected from the group consisting of glucose, cortisol, creatinine, lactate, C-reactive protein, alpha-fetoprotein, cardiac troponin I (cTnI), cardiac troponin T (cTNT), cardiac phosphocreatine kinase M and B (CK-MB), brain natriuretic peptide (BNP), or any combination thereof.

27. The device of claim 26, wherein the analyte is cortisol.

28. The device of claim 24, wherein the sample is diluted 1:1 by volume with a colloidal gold solution (OD=2) before addition to the one or more LSPR sensors.

29. The device of claim 28, wherein the colloidal gold is coated with both an anti-analyte antibody and alkaline phosphatase.

30. The device of claim 29, wherein BCIP/NBT is used as a substrate for alkaline phosphatase.

31. The device of claim 23, wherein the presence of the analyte in the sample is detected by means of a shift in the wavelength of light reflected from the one or more LSPR sensor surfaces.

32. The device of claim 24, wherein a limit of detection for the competitive immunoassay performed in the device is better than about 1,000 pg/mL.

33. The device of claim 24, wherein a limit of detection for the competitive immunoassay performed in the device is better than about 100 pg/mL.

34. The device of claim 24, wherein a limit of detection for the competitive immunoassay performed in the device is better than about 10 pg/mL.

35. The device of claim 24, wherein a limit of detection for the competitive immunoassay performed in the device is better than about 1 pg/mL.

36. The device of claim 23, wherein the substrate comprises two or more LSPR sensors, and wherein at least one of the LSPR sensors is used to perform a control.

37. The device of claim 23, wherein the sample is saliva.

38. The device of claim 37, wherein the saliva is human saliva.

39. The device of claim 23, wherein the sample is blood plasma or serum.

40. The device of claim 23, wherein the cartridge comprises one or more reaction wells comprising the one or more LSPR sensors, and wherein the surface(s) of the one or more LSPR sensors are accessible to addition of the sample by pipetting the sample into the one or more reaction wells.

41. The device of claim 23, wherein the cartridge comprises a sample reservoir and one or more reaction chambers comprising the one or more LSPR sensors, and the surface(s) of the one or more LSPR sensors are accessible to addition of the sample by flowing the sample from the sample reservoir to each of the one or more reaction chambers via interconnecting fluid channels.

42. The device of claim 41, wherein the one or more reaction chambers are arranged in a hub-and-spoke pattern around a central sample reservoir.

43. The device of claim 42, wherein the sample is caused to flow from the sample reservoir to each of the one or more reaction chambers via interconnecting fluid channels by exerting pressure on the sample reservoir using a mechanical piston.

44. The device of claim 41, wherein the cartridge further comprises one or more valves for controlling the flow of sample or other fluids between the sample reservoir and the one or more reaction chambers.

45. The device of claim 41, wherein the cartridge further comprises one or more reagent wells that are interconnected with the sample reservoir and the one or more reaction chambers via fluid channels.

46. The device of claim 45, wherein the one or more reagent wells comprise pre-packaged assay reagents and/or controls.
